(19) United States
(12) Patent Application Publication
(10) Pub. No.: US 2007/0292416 A1
(43) Pub. Date: Dec. 20, 2007

(54) De-Immunized Anti-Cd3 Antibody

(75) Inventors: Russell P. Rother, Prospect, CT (US); Susan Faas McKnight, Old Lyme, CT (US); Dayang Wu, Cheshire, CT (US); Francis J. Carr, Aberdeenshire (GB); Anita Hamilton, Aberdeen (GB)

Correspondence Address: ROPES & GRAY LLP;PATENT DOCKETING 39/41, ONE INTERNATIONAL PLACE, BOSTON, MA, 02110-2624, US

(73) Assignee: Alexion Pharmaceuticals, Inc., Cheshire, CT (US)

(21) Appl. No.: 10/559,543

(22) PCT Filed: May 28, 2004
(86) PCT No.: PCT/US04/17219

§ 371 Date: Feb. 26, 2007

Related U.S. Application Data
(60) Provisional application No. 60/475,155, filed on Jun. 2, 2003.

(51) Int. Cl. A61K 39/395; A61P 37/00; C07H 21/02; C07K 16/00; C12N 15/00; C12P 21/00

(52) U.S. Cl. 424/131.1; 435/320.1; 435/69.6; 530/387.1; 536/23.1

(57) ABSTRACT

Antibodies or functional antibody fragments that recognize or interfere with the production of a component of the CD3 antigen complex are de-immunized.

### RELATED APPLICATIONS

This application claims priority to U.S. Provisional Application 60/475,155 filed Jun. 2, 2003, the entire disclosure of which is incorporated herein by this reference.

### BACKGROUND

1. Technical Field

The present disclosure relates to the field of genetically engineered antibodies. More specifically this disclosure relates to anti-CD3 antibodies which have been structurally altered to eliminate binding to HLA proteins, thereby potentially reducing immunogenicity.

2. Background of Related Art

Antibodies are produced by B lymphocytes and defend against infections. The basic structure of an antibody consists of two identical light polypeptide chains and two identical heavy polypeptide chains linked together by disulphide bonds. The first domain located at the amino terminus of each chain is highly variable in amino acid sequence, providing the vast spectrum of antibody binding specificities found in each individual. These are known as variable heavy (VH) and variable light (VL) regions. The other domains of each chain are relatively invariant in amino acid sequence and are known as constant heavy (CH) and constant light (CL) regions.

The interaction between the antigen and the antibody takes place by the formation of multiple bonds and attractive forces such as hydrogen bonds, electrostatic forces and Van der Waals forces. Together these form considerable binding energy which allows the antibody to bind the antigen. Antibody binding affinity and avidity have been found to affect the physiological and pathological properties of antibodies.

The advent of genetic engineering technology has led to various means of producing unlimited quantities of uniform antibodies (monoclonal antibodies) which, depending upon the isotype, exhibit varying degrees of effector function. For example, certain murine isotypes (IgG1, IgG2) as well as human isotypes (particularly IgG1) can effectively bind to Fc receptors on cells such as monocytes, B cells and NK cells, thereby activating the cells to release cytokines. Such antibody isotypes are also potent in activating complement, with local or systemic inflammatory consequences. The anti-CD3 murine antibody OKT3 is one antibody that has been observed to cause significant cytokine release leading to cytokine release syndrome (CRS). The human CD3 antigen consists of at least four invariant polypeptide chains, which are non-covalently associated with the T cell receptors (TCR) on the surface of T-cells, typically referred to as the CD3 antigen complex. The CD3 antigen complex plays an important role in the T-cell activation upon antigen binding to the T cell receptor. Some anti-CD3 antibodies can activate T-cells in the absence of antigen-TCR ligation, but such activation also depends on the interaction of the Fc portion of the mAb and the Fc receptors on accessory cells to crosslink CD3 complexes on the T-cells. The importance of Fc interactions in anti-CD3 mediated T-cell activation is illustrated by the observation that “mitogenic” anti-CD3 antibodies such as OKT3 do not stimulate T-cells to proliferate in vitro unless they are bound to plastic (which permits CD3 cross-leaking) or bound to Fc receptor bearing cells.

Antibodies to the CD3 ε signaling molecule of the T-cell receptor complex have proven to be powerful immunosuppressants. For example, OKT3 is a mouse IgG2a/k MAb which recognizes an epitope on the T-cell receptor-CD3 epsilon chain and has been approved for use in many countries throughout the world as an immunosuppressant in the treatment of acute allograft rejection. The binding of OKT3 to CD3 results in a coating and/or modulation of the entire TcR complex, which mediates TcR blockade and may be one mechanism by which alloantigen and cell-mediated cytotoxicity are inhibited.

The murine OKT3 antibody has been in use therapeutically since its approval in 1985. However, in view of the murine nature of this MAb, a significant human anti-mouse antibody (HAMA) response, with a major anti-idiotype component occurs, which severely limits the dosing potential of this antibody. A HAMA response is initiated when T cells from an individual make an immune response to the administered antibody. The T cells then recruit B cells to generate specific “anti-antibody” antibodies. Thus the HAMA response, though mediated by B cell-generated antibodies directed against mouse antibodies, depends upon an initial T cell response to occur. Clearly, it would be highly desirable to diminish or abolish this HAMA response by suitable humanization or other recombinant DNA manipulation of this very useful antibody and thus enlarge its area of use.

Several techniques have been employed to address the HAMA problem and thus enable the use of therapeutic murine-derived monoclonal antibodies in humans. A common aspect of these methodologies has been the introduction into the therapeutic antibody, which in general is of rodent origin, of significant tracts of sequence identical to that present in human antibody proteins. Such alterations are also usually coupled to alteration of particular single amino acid residues at positions considered critical to maintaining the antibody-antigen binding interaction. For antibodies, this process is possible due to the very high degree of structural (and functional) conservation between antibody molecules of different species. However for potentially therapeutic proteins where no structural homologue may exist in the host species (e.g. human) for the therapeutic protein, such processes are not applicable.

The term humanized antibody describes a molecule having certain components of the antigen binding site called complementarity determining regions (CDRs) derived from an antibody from a non-human species, while the remaining regions of the antigen binding site (called framework regions) are derived from human antibodies. The antigen binding site may also comprise complete non-human variable regions fused onto human constant domains (a “chimeric” antibody). Since a primary function of an antibody is to bind its target antigen, it is important that the original features of the antibody are preserved in such a way that the antigen specificity and affinity are maintained. Unfortunately, however, humanization of non-human antibodies has unpredictable effects on antibody-antigen interactions, e.g., antigen binding properties. This means that in therapeutic applications, more of the humanized antibody may be required per dose resulting in a higher cost of treatment and potentially greater risk of adverse events. In addition, both fully human and humanized antibodies can provoke an immune response or be immunogenic when administered to certain individuals.

According to another method, an antibody is rendered non-immunogenic, or less immunogenic, to a given species, by first determining at least part of the amino acid sequence of the protein and then identifying in that amino acid sequence one or more potential epitopes to which T cells from the given species can react. Next, the amino acid sequence of the antibody is modified to eliminate at least one of the T cell epitopes identified to reduce the immunogenicity of the protein or part thereof when exposed to the immune system of the given species. Unlike antibodies, which can recognize and bind to soluble antigens, T cells must encounter their antigen targets through the activity of specialized antigen-presenting cells (APC's) such as dendritic cells and macrophages. APC's ingest foreign antigens and process them into peptides, which are then complexed to the HLA proteins and expressed on the surface of the APC. T cells can only recognize antigen fragments “presented” in the context of HLA. In the method which has been termed “de-immunization” and is described herein, amino acids within the antibody sequence that are predicted to bind effectively to HLA molecules are changed such that they no longer bind HLA and thus can no longer stimulate a T cell response. The lack of a T cell response to antigen translates into a reduction or elimination of a HAMA response.

### SUMMARY

Antibodies in accordance with this disclosure recognize the CD3 antigen complex or interfere with the cell-surface expression of a component of the CD3 antigen complex. The anti-CD3 antibodies are also de-immunized (that is, rendered non-immunogenic, or less immunogenic, to a given species). In a particularly useful embodiment, de-immunization is achieved by first determining at least part of the amino acid sequence of the protein and then identifying in the amino acid sequence one or more potential epitopes for T cells (“T cell epitopes”) which are able to bind to HLA proteins of the given species. Next, the amino acid sequence of the antibody is modified to eliminate at least one of the T cell epitopes identified in order to reduce the immunogenicity of the protein or part thereof when exposed to the immune system of the given species.

In another aspect, this disclosure relates to a process for producing an antibody which includes the steps of: (a) producing an expression vector having a DNA sequence which includes a sequence that encodes an anti-CD3 antibody, at least a portion of which has been de-immunized; (b) transfecting a host cell with the vector; and (c) culturing the transfected cell line to produce the engineered antibody molecule.

### BRIEF DESCRIPTION OF THE DRAWINGS

FIGS. 1A and 1B show the complete nucleotide and amino acid sequences of the OKT3 heavy chain variable region (GenBank Accession number A22261) and light chain variable region (GenBank Accession number A22259), respectively.

FIG. 2 schematically shows expression cassettes for the heavy and light chain variable regions as HindIII to BamH1 fragments.

FIG. 3 shows the complete nucleotide (SEQ ID NO: 1) and amino acid sequences of the murine OKT3 heavy chain variable region, including the murine immunoglobulin promoter, a murine signal sequence with intron at the 5′ ends, and a splice donor site (Bam HI) at the 3′ ends. Restriction enzyme sites are indicated.

FIG. 4 shows the complete nucleotide (SEQ ID NO: 3) and amino acid sequences of the murine OKT3 light chain variable region, including the murine immunoglobulin promoter, a murine signal sequence with intron at the 5′ ends, and a splice donor site (Bam HI) at the 3′ ends. Restriction enzyme sites are indicated.

FIG. 5A shows a graphic map of the vector APEX-1 3F4V_{H}HuGamma4.

FIG. 5B shows the complete nucleotide sequence of the vector (SEQ ID NO: 5) and indicates the amino acid and nucleotide sequences of the hIgG4 insert adjacent to an irrelevant VH region (labeled 3F4VH). The locations of the signal sequence, CH1, hinge, CH2 and CH3 regions are indicated.

FIG. 6A shows a graphic map of the vector APEX-1 3F4V_{H}HuG2/G4.

FIG. 6B shows the nucleotide sequence of the vector (SEQ ID NO: 7) and the amino acid and nucleic acid sequence of the G2/G4 insert, and indicates the locations of the signal sequence, irrelevant Vh (herein labeled 3F4Vh), CH1, hinge, CH2 and CH3 regions.

FIG. 7 shows a graphic map of the heavy chain expression vector pSVgptHuG2/G4.

FIG. 8 shows the complete nucleotide sequence (SEQ ID NO: 9) of the HuG2/G4 fragment excised from the APEX-1 3F4V_{H}HuG2/G4 vector and modified for insertion into a PUC 19 cloning vector by the addition, at the 5′ end, of a Bam HI site and 5′ untranslated intron sequences from native human IgG4 and, at the 3′ end, of a Bgl II site and 3′ untranslated sequence from natural human IgG4.

FIG. 9 shows a graphic map of the expression vector pSVgptHuCk and indicates the position of the light chain variable and constant regions.

FIG. 10 shows the amino acid sequences of the murine OKT3 variable heavy chain (SEQ ID NO: 10) and the amino acid sequences of several of the deimmunized heavy chain variable regions (SEQ ID NOS: 11-17) constructed in the Example.

FIG. 11 shows the amino acid sequences of the murine OKT3 variable light chain (SEQ ID NO: 18) and the amino acid sequences of two deimmunized light chain variable regions (SEQ ID NOS: 19 and 20) constructed in the Example.

FIG. 12 shows the mutagenic oligonucleotides primers used to construct the designed de-immunized sequences by mutagenesis using overlapping PCR.

FIG. 13 shows the nucleic acid (SEQ ID NO: 21) and amino acid sequences for the de-immunized VH expression cassette OKT3DIVHV1.

FIG. 14 shows the nucleic acid (SEQ ID NO: 23) and amino acid sequences for the de-immunized Vκ expression cassette OKT3DIVKV1

FIG. 15 shows binding of murine OKT3 and chimeric OKT3 to Jurkat, JRT3 and HPB-ALL cells.

FIGS. 16 and 17 are tables showing the binding of de-immunized anti-CD3 antibodies to HPB-ALL and JRT3 CELLS.

FIGS. 18, 19, 20 and 21 show the results of competition assays measuring the affinity of the de-immunized antibodies relative to that of chimeric OKT3 and murine OKT3.

FIG. 22 is a table summarizing the IC50 of the de-immunized antibodies relative to that of murine OKT3.

### DETAILED DESCRIPTION OF THE PREFERRED EMBODIMENTS

De-immunized anti-CD3 antibodies are described. The term “anti-CD3 antibodies” means any antibody or functional antibody fragment that recognizes the CD3 antigen complex or interferes with the cell surface expression of a component of the CD3 antigen complex. The anti-CD3 antibody can be recombinant or naturally occurring. The anti-CD3 antibody can be human, non-human, chimeric or humanized. Anti-CD3 antibodies are known to those skilled in the art and include, for example, the antibodies described in U.S. Pat. No. 5,527,713 entitled “Methods for inducing a population of T cells to proliferate using agents which recognize TCR/CD3 and ligands which stimulate an accessory molecule on the surface of the T cells”; U.S. Pat. No. 6,352,694 entitled “Methods for inducing a population of T cells to proliferate using agents which recognize TCR/CD3 and ligands which stimulate an accessory molecule on the surface of the T cells”; U.S. Pat. No. 6,406,696 entitled “Methods of stimulating the immune system with anti-CD3 antibodies”; U.S. Pat. No. 6,143,297 entitled “Methods of promoting immunopotentiation and preparing antibodies with anti-CD3 antibodies”; U.S. Pat. No. 6,113,901 entitled “Methods of stimulating or enhancing the immune system with anti-CD3 antibodies”; U.S. Pat. No. 6,491,916 entitled “Methods and materials for modulation of the immunosuppresive activity and toxicity of monoclonal antibodies”; U.S. Pat. No. 5,929,212 entitled “CD3 specific recombinant antibody”; U.S. Pat. No. 5,834,597 entitled “Mutated nonactivating IgG2 domains and anti CD3 antibodies incorporating the same”; U.S. Pat. No. 5,527,713 entitled “Anti-CD3 antibody-aminodextran conjugates for induction of T-cell activation and proliferation”; U.S. Pat. No. 5,316,763 entitled “Short-term anti-CD3 stimulation of lymphocytes to increase their in vivo activity”; U.S. Pat. No. 5,821,337 entitled “Immunoglobulin variants”. Each of these patents is incorporated herein in its entirety by this reference.

The anti-CD3 antibody is de-immunized. De-immunization renders the anti-CD3 antibody non-immunogenic, or less immunogenic, to a given species. De-immunization can be achieved through structural alterations to the anti-CD3 antibody. Any de-immunization technique known to those skilled in the art can be employed. One suitable technique for de-immunizing antibodies is described, for example, WO 00/34317 published Jun. 15, 2000, the disclosure of which is incorporated herein in its entirety. In summary, a typical protocol within the general method described therein includes the following steps.

1. Determining the amino acid sequence of the antibody or a part thereof (if modification of only of a part is required);

2. Identifying potential T cell epitopes within the amino acid sequence of the antibody by any method including determination of the binding of peptides to MHC molecules, determination of the binding of peptide:HLA complexes to the T cell receptors from the species to receive the therapeutic protein, testing of the antibody or parts thereof using transgenic animals with HLA molecules of the species to receive the therapeutic protein, or testing such transgenic animals reconstituted with immune system cells from the species to receive the therapeutic protein;

3. By genetic engineering or other methods for producing modified antibodies, altering the antibody to remove one or more of the potential T cell epitopes and producing such an altered antibody for testing;

4. Optionally within step 3, altering the antibody to remove one or more of the potential B cell epitopes;

5. Testing altered antibodies with one or more potential T cell epitopes (and optionally B cell epitopes) removed in order to identify a modified antibody which has retained all or part of its desired activity but which has lost one or more T cell epitopes. Potential T-cell epitopes herein are defined as specific peptide sequences which either are predicted to or that bind with reasonable efficiency to HLA class II molecules (or their equivalent in a non-human species), or which in the form of peptide:HLA complexes bind strongly to the T cell receptors from the species to receive the therapeutic protein or which, from previous or other studies, show the ability to stimulate T-cells via presentation on HLA class II molecules present on antigen presenting cells from the species to receive the therapeutic antibody.

This de-immunization method recognizes that an effective T cell-dependant immune response to a foreign protein requires activation of the cellular arm of the immune system. Such a response requires the uptake of the therapeutic (foreign) protein (i.e. therapeutic antibody) by antigen presenting cells (APCs). Once inside such cells, the protein is processed and fragments of the protein form a complex with MHC, class II molecules and are presented at the cell surface. Should such a complex be recognized by binding of the T cell receptor from T-cells, such cells can be, under certain conditions, activated to produce stimulatory cytokines. The cytokines will elicit differentiation of B-cells to mature antibody producing cells. In addition, such T cell responses may also mediate other deleterious effects on the patient such as inflammation and possible allergic reaction.

The whole anti-CD3 antibody or only a portion thereof (e.g., the variable portions of the anti-CD3 antibody) can be de-immunized. De-immunization of only a portion of the anti-CD3 antibody is particularly useful where the anti-CD3 antibody is a chimeric antibody (e.g. one with human constant regions).

The term “antibody” as used herein includes whole polyclonal and monoclonal antibodies, single chain antibodies, and other functional antibody fragments. Whole, monoclonal antibodies are preferred.

In general, the construction of the antibodies disclosed herein is achieved by using recognized manipulations utilized in genetic engineering technology. For example, techniques for isolating DNA, making and selecting vectors for expressing the DNA, purifying and analyzing nucleic acids, specific methods for making recombinant vector DNA (e.g. PCR), cleaving DNA with restriction enzymes, ligating DNA, introducing DNA, including vector DNA, into host cells by stable or transient means, culturing the host cells in selective or non-selective media, to select and maintain cells that express DNA, are generally known in the field.

The monoclonal antibodies disclosed herein may be derived using the hybridoma method (Kohler et al., Nature, 256:495, 1975), or other recombinant DNA methods well known in the art. In the hybridoma method, a mouse or other appropriate host animal is immunized with a protein which elicits the production of antibodies by the lymphocytes. Alternatively, lymphocytes may be immunized in vitro. The lymphocytes produced in response to the antigen are then are fused with myeloma cells using a suitable fusing agent, such as polyethylene glycol, to form a hybridoma cell (Goding, Monoclonal Antibodies: Principles and Practice, pp. 59-103 (Academic Press, 1986). The hybridoma cells are then seeded and grown in a suitable culture medium that preferably contains one or more substances that inhibit the growth or survival of the unfused, parental myeloma cells. Preferred myeloma cells are those that fuse efficiently, support stable production of antibody by the selected antibody-producing cells, and are not sensitive to a selective medium such as HAT medium (Sigma Chemical Company, St. Louis, Mo., Catalog No. H-0262). Among these, preferred myeloma cell lines are murine myeloma lines, such as those derived from MOPC-21 and MPC-11 mouse tumors available from the Salk Institute Cell Distribution Center, San Diego, Calif. USA, and SP-20, NS0 or X63-Ag8-653 cells available from the American Type Culture Collection, Rockville, Md. USA.

The hybridoma cells are grown in a selective culture medium (e.g., HAT) and surviving cells expanded and assayed for production of monoclonal antibodies directed against the antigen. The binding specificity of monoclonal antibodies produced by hybridoma cells may be determined by assays, such as, immunoprecipitation, radioimmunoassay (RIA), flow cytometry or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986). In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, or mammalian cells that do not otherwise produce immunoglobulin protein, to obtain the synthesis of monoclonal antibodies in the recombinant host cells.

Antibodies or antibody fragments can also be isolated from antibody phage libraries generated using the techniques described in McCafferty et al., Nature, 348:552-554 (1990). Other publications have described the production of high affinity (nM range) human antibodies by chain shuffling (Marks et al., Bio/Technology, 10:779-783, 1992), as well as combinatorial infection and in vivo recombination as a strategy for constructing very large phage libraries (Waterhouse et al., Nuc. Acids. Res., 21:2265-2266, 1993). Thus, these techniques are viable alternatives to traditional monoclonal antibody hybridoma techniques for isolation of antigen-specific monoclonal antibodies.

In another aspect, this disclosure provides recombinant expression vectors which include the synthetic, genomic, or cDNA-derived nucleic acid fragments necessary to produce a de-immunized anti-CD3 antibody. The nucleotide sequence coding for any de-immunized anti-CD3 antibody in accordance with this disclosure can be inserted into an appropriate vector which contains the necessary elements for the transcription and translation of the inserted protein-coding sequence. Any suitable host cell vector may be used for expression of the DNA sequences coding for the de-immunized anti-CD3 antibody. Bacterial (e.g. E. coli) and other microbial systems may be used. Eukaryotic (e.g. mammalian) host cell expression systems may also be used to obtain antibodies of the present disclosure. Suitable mammalian host cell include COS cells and CHO cells (Bebbington C R (1991) Methods 2 136-145); and myeloma or hybridoma cell lines (for example NSO cells; Bebbington, et al., Bio Technology, 10, 169-175, 1992).

The de-immunized anti-CD3 antibodies can also be used as separately administered compositions given in conjunction with therapeutic agents. For diagnostic purposes, the antibodies may either be labeled or unlabeled. Unlabeled antibodies can be used in combination with other labeled antibodies (second antibodies) that are reactive with the de-immunized anti-CD3 antibody, such as antibodies specific for human immunoglobulin constant regions. Alternatively, the de-immunized antibodies can be directly labeled. A wide variety of labels may be employed, such as radionuclides, fluors, enzymes, enzyme substrates, enzyme co-factors, enzyme inhibitors, ligands (particularly haptens), etc. Numerous types of immunoassays are available and are well known to those skilled in the art.

The present de-immunized anti-CD3 antibodies can be administered to a patient in a composition comprising a pharmaceutical carrier. A pharmaceutical carrier can be any compatible, non-toxic substance suitable for delivery of the antibodies to the patient. Sterile water, alcohol, fats, waxes, and inert solids may be included in the carrier. Pharmaceutically accepted adjuvants (buffering agents, dispersing agent) may also be incorporated into the pharmaceutical composition.

The antibody compositions may be administered to a patient in a variety of ways. Preferably, the pharmaceutical compositions may be administered parenterally (e.g., subcutaneously, intramuscularly or intravenously). Thus, compositions for parental administration may include a solution of the antibody, antibody fragment or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of antibody or antibody fragment in these formulations can vary widely, e.g., from less than about 0.5%, usually at or at least about 1% to as much as 15 or 20% by weight and will be selected primarily based on fluid volumes, viscosities, etc., in accordance with the particular mode of administration selected.

Actual methods for preparing parenterally administrable compositions and adjustments necessary for administration to subjects will be known or apparent to those skilled in the art and are described in more detail in, for example, Remington's Pharmaceutical Science, 17^{th} Ed., Mack Publishing Company, Easton, Pa. (1985), which is incorporated herein by reference.

The following examples are intended to illustrate but not limit the invention. While they are typical of those that might be used, other procedures known to those skilled in the art may alternatively be used.

### EXAMPLE 1

De-immunized, Chimeric Anti-CD3 Antibody

A de-immunized, chimeric anti-CD3 antibody was prepared. The variable regions selected were derived from the known mouse anti-human CD3 antibody OKT3. The variable regions were de-immunized and combined with an engineered human constant region to prepare the chimeric, de-immunized anti-CD3 antibody. The procedures used to prepare and test the chimeric, de-immunized anti-CD3 antibodies are described below.

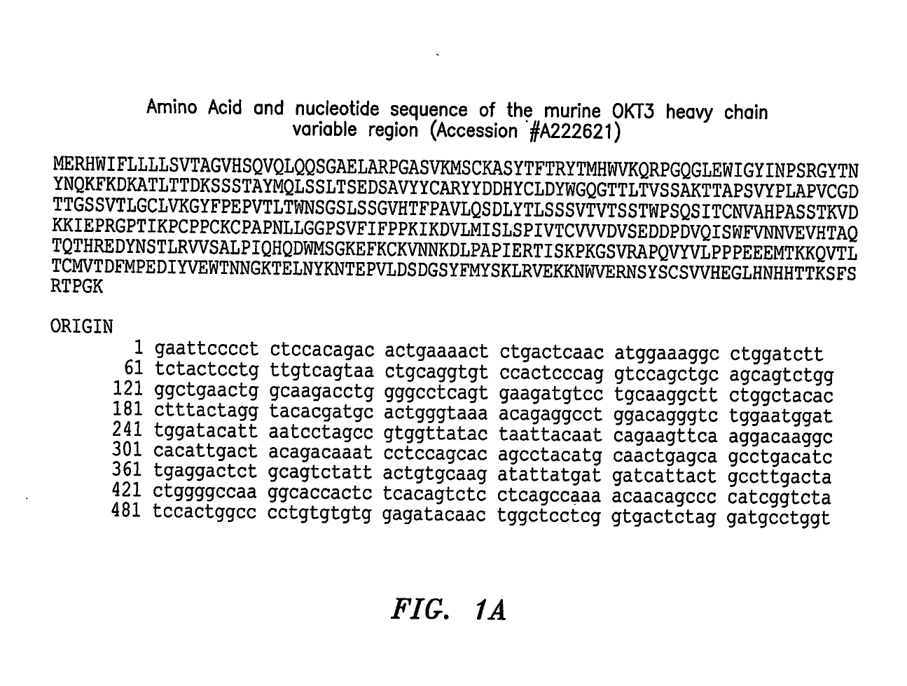

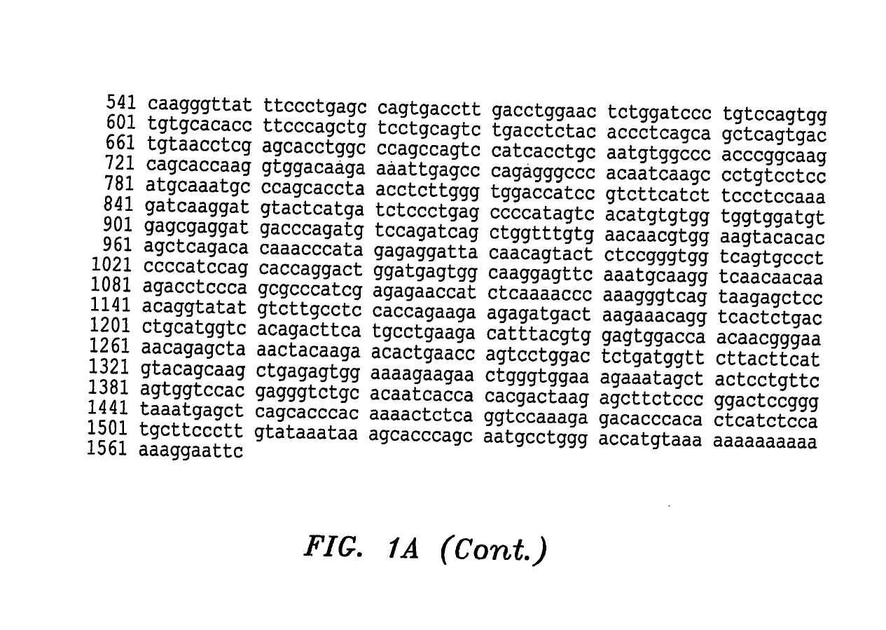

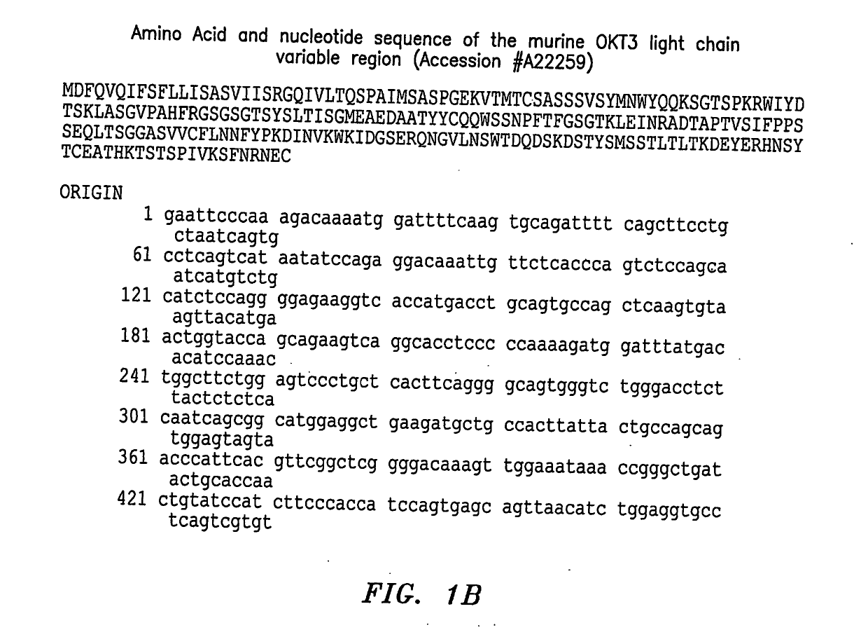

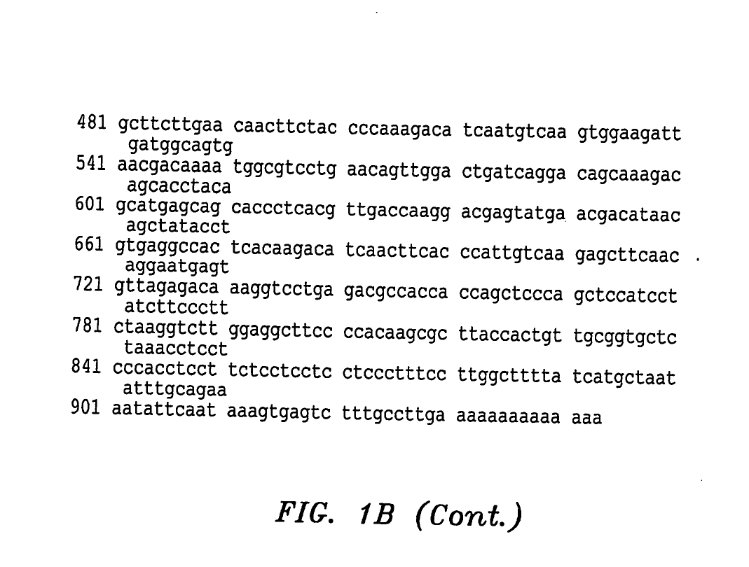

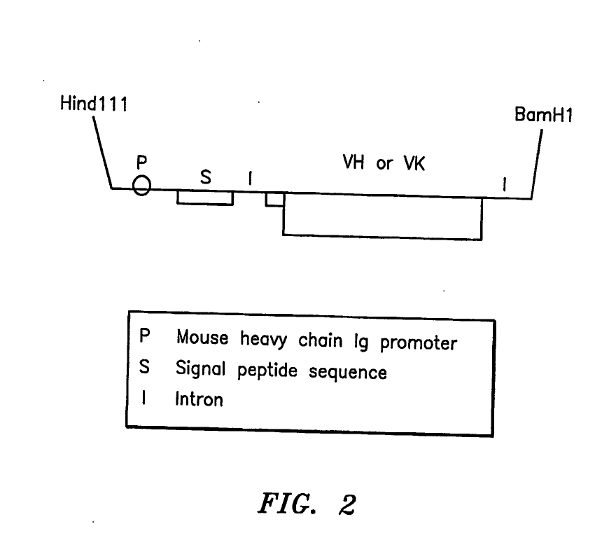

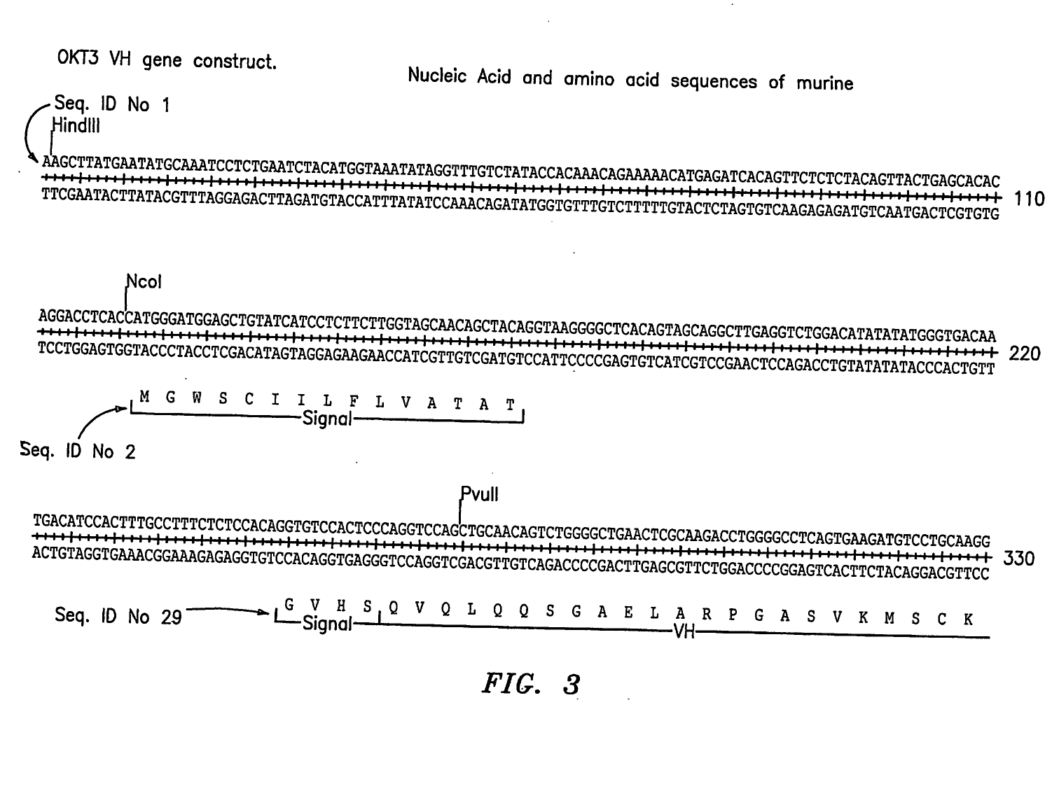

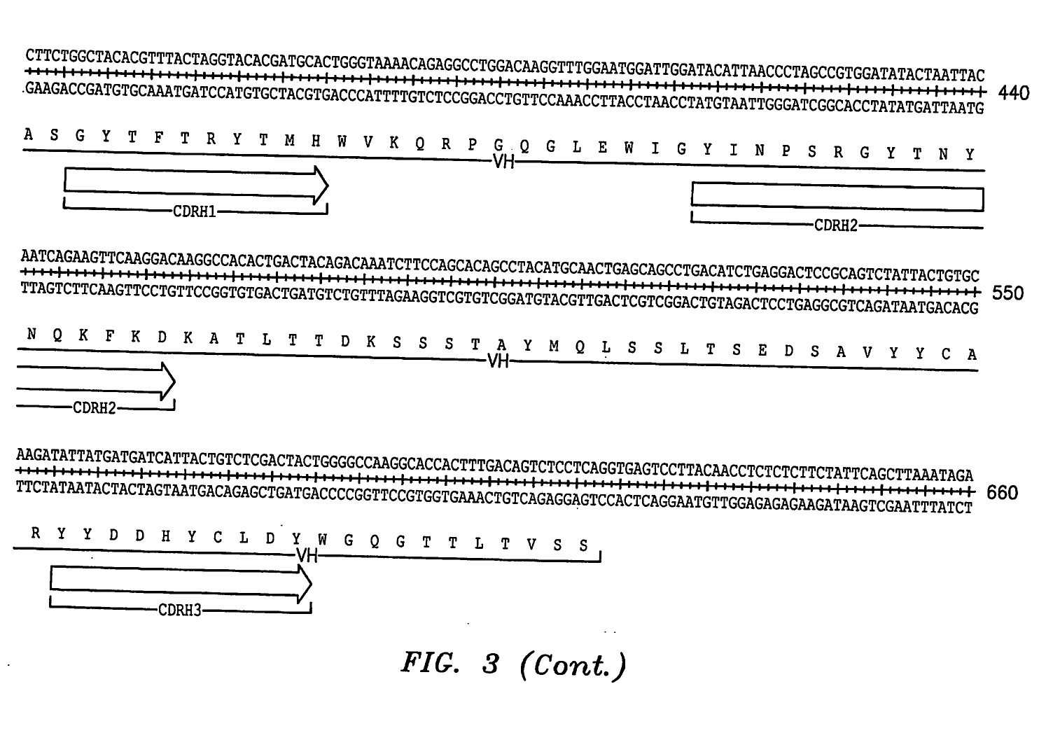

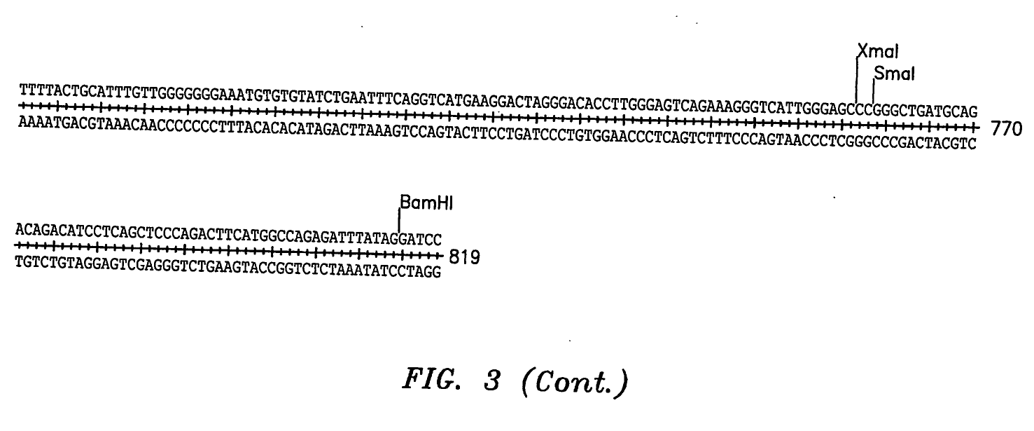

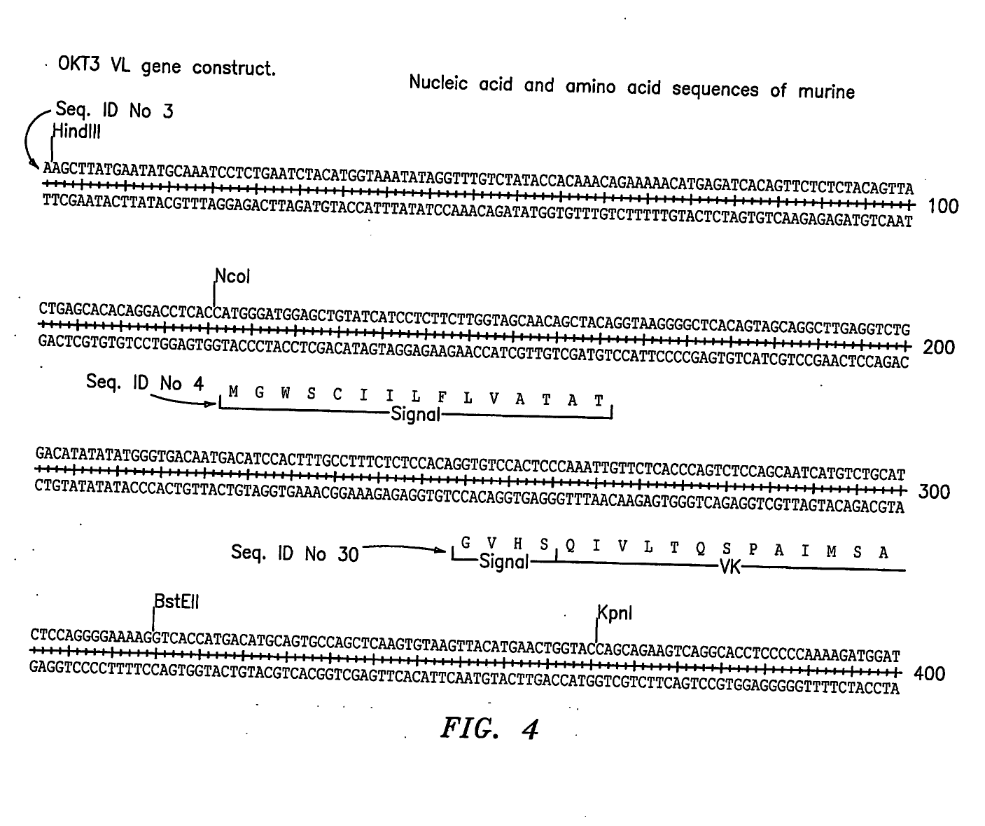

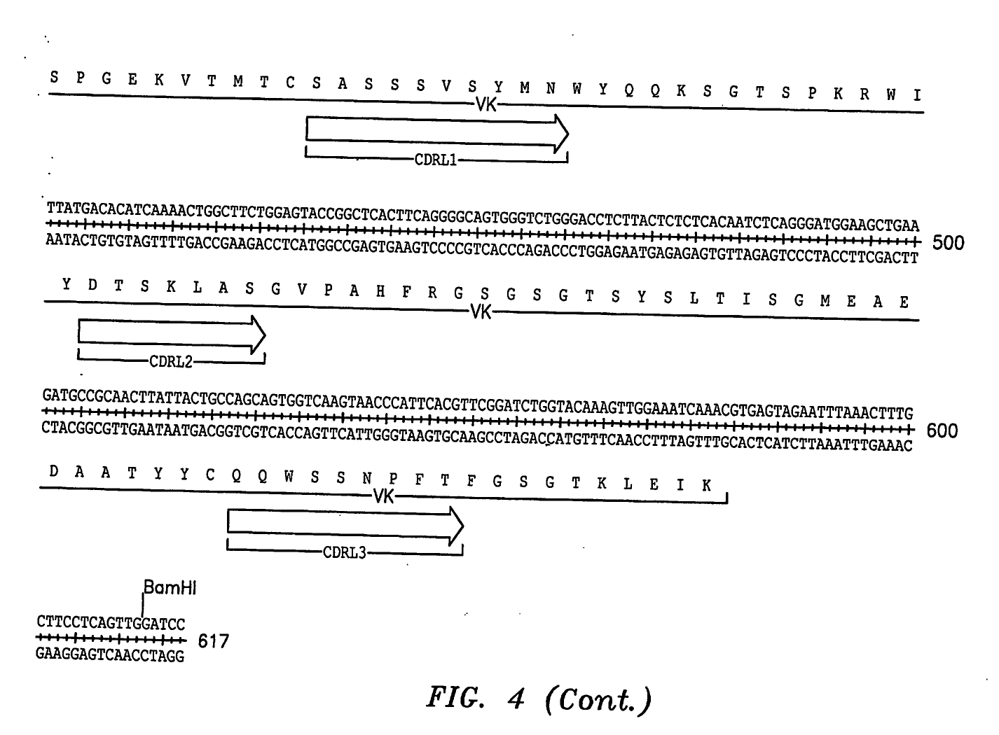

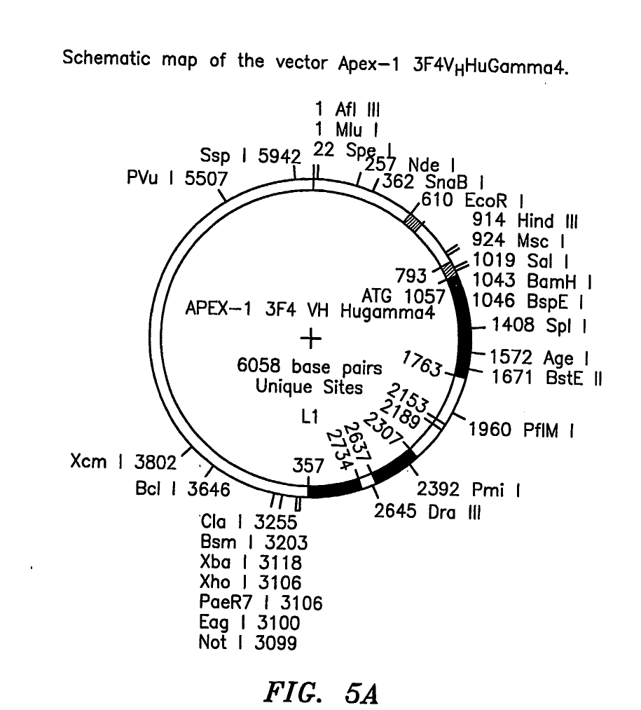

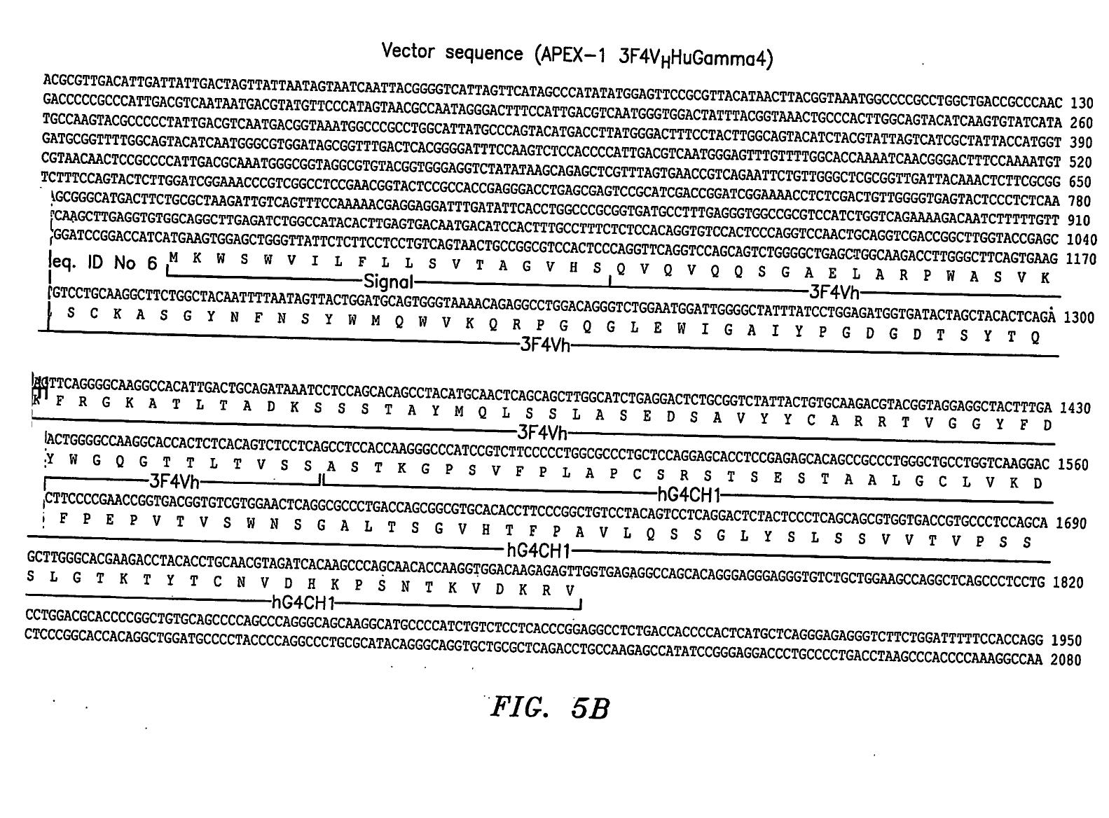

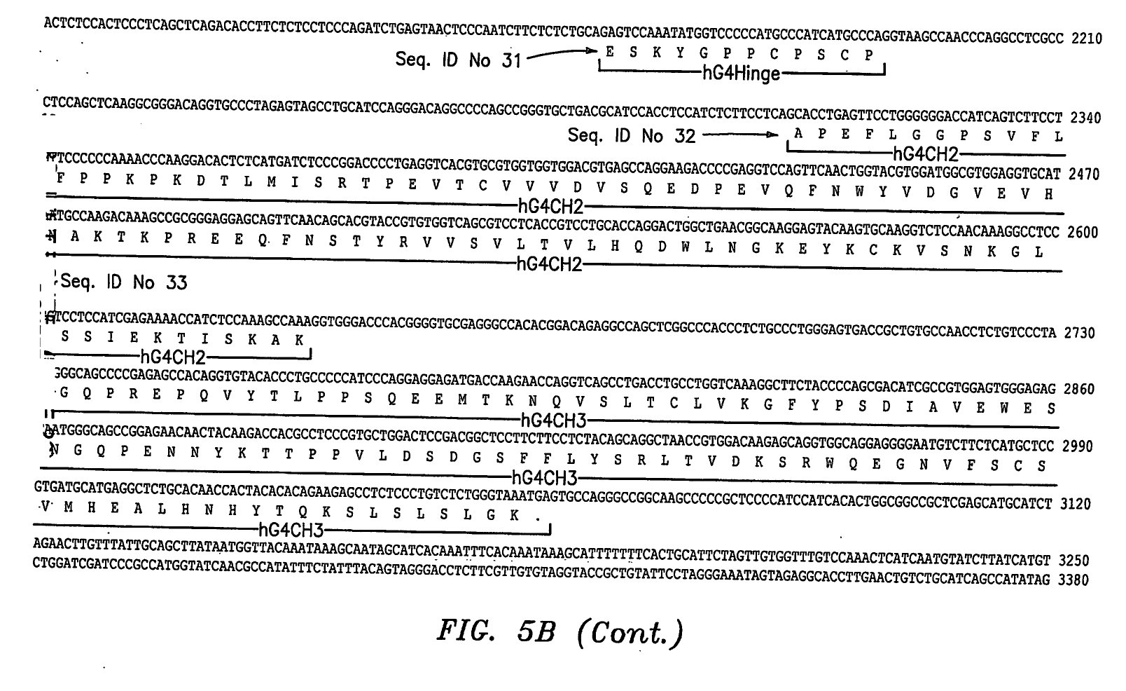

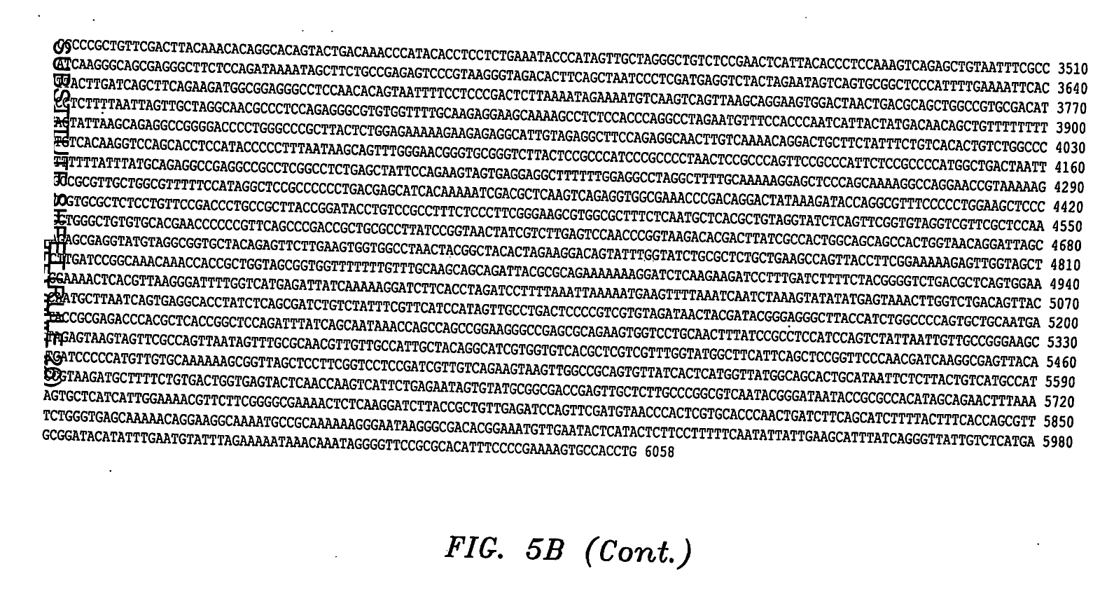

The murine OKT3 heavy and light chain variable regions were constructed synthetically by gene synthesis using overlapping 40 mer oligonucleotides and a polymerase chain reaction. The sequences of the heavy and light chain variable regions of this antibody have been previously determined and deposited in the GenBank database (Accession numbers A22261 and A22259 respectively; see FIG. 1). Sequences, including the murine immunoglobulin promoter and a murine signal sequence with intron, were added at the 5′ ends, and sequences including the splice donor site were added at the 3′ end by PCR to form expression cassettes (see FIG. 2) for the heavy and light chain variable regions as HindIII to BamHI fragments. The entire sequences of the expression cassettes were confirmed to be correct. The complete DNA and amino acid sequences of the murine OKT3 heavy and light chain expression cassettes are shown in FIGS. 3 and 4, respectively. The heavy chain constant region was engineered to include a human IgG2 portion and a human IgG4 portion (“HuG2G4 constant region”). This constant region was prepared as follows: First, the genomic DNA encoding the human IgG4-derived portions (part of CH2 and CH3 regions) was inserted into the bacterial carrier plasmid pBR322, a plasmid derived from an E. coli species (ATCC 37017; Mandel, M. et al. (1970) J. Mol. Biol. 53, 154). The IgG4-derived insert was released from the plasmid by performing a restriction digest with Hind III and Xho I. The insert was gel purified, excised, and subjected to further restriction analysis to confirm the published sequence of the human IgG4 genomic DNA. The individual genomic IgG4 insert (HindIII/SmaI restriction fragment; the SmaI site is in the 3′ untranslated region approximately 30 bp 3′ of the translation stop site for each insert) was then subcloned by ligation into the expression cassette APEX-1 to yield APEX-1 3F4 VH HuGamma 4 (see FIG. 5). DNA sequence analysis was performed to confirm the correct sequence of the human IgG4 desired regions.

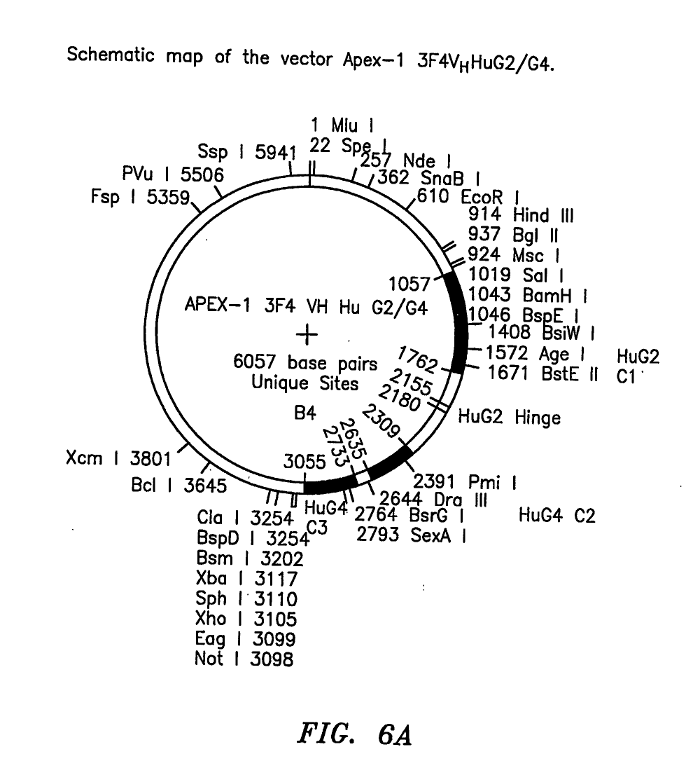

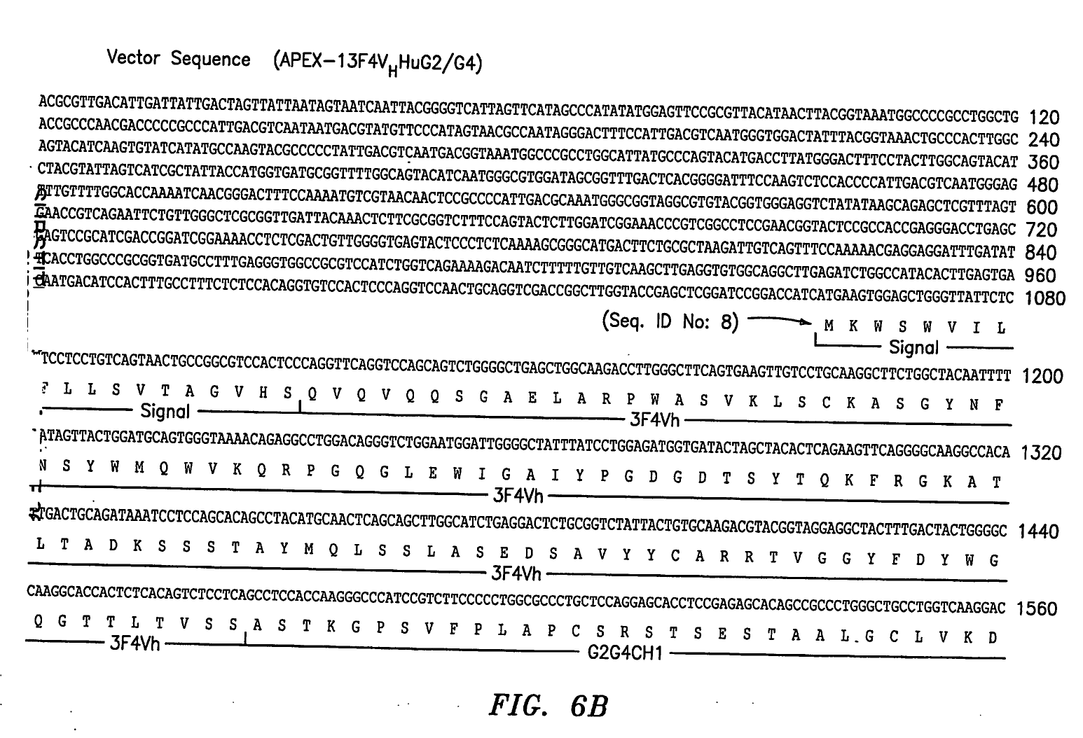

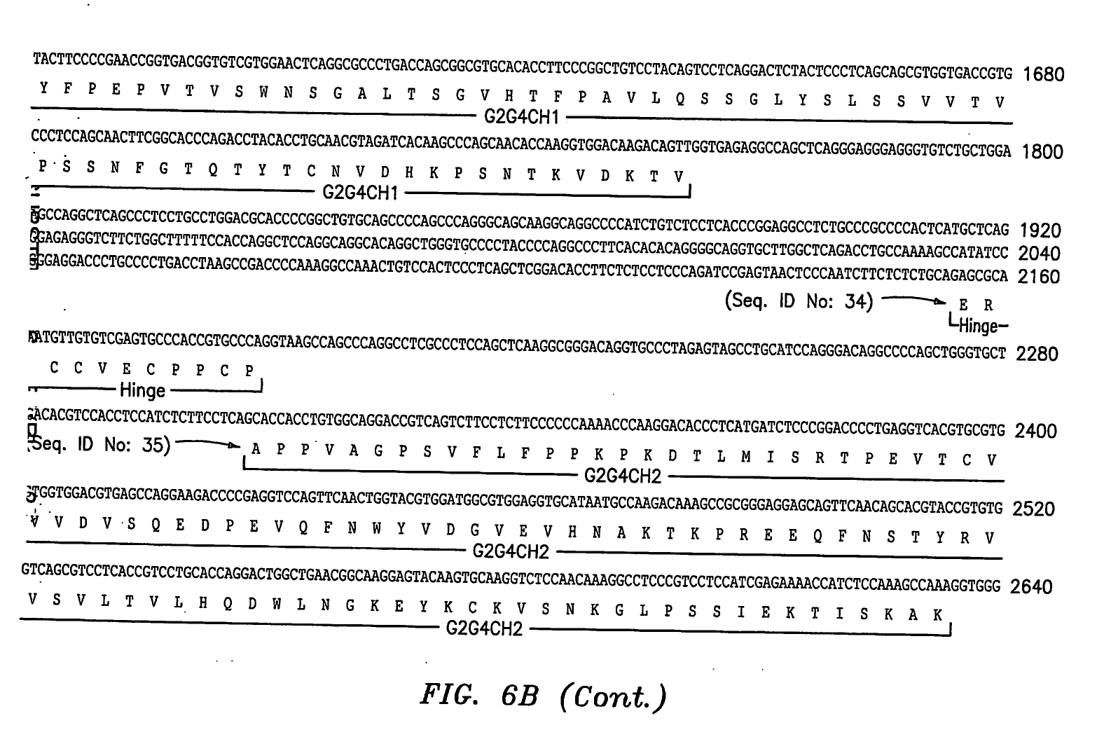

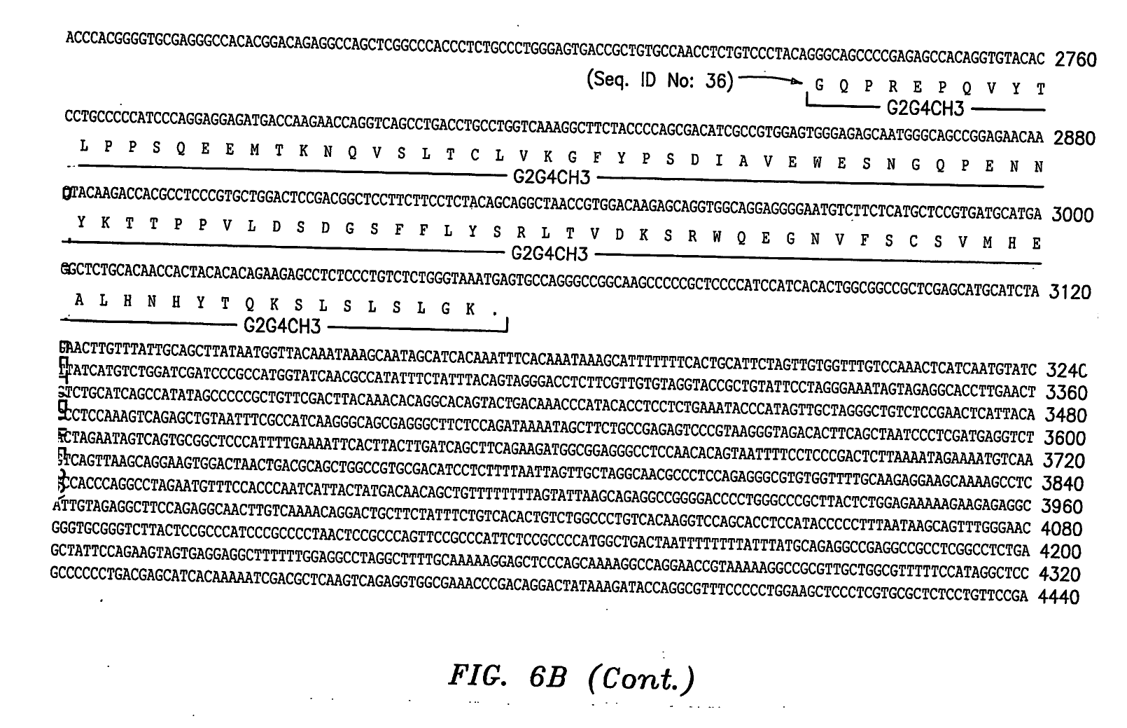

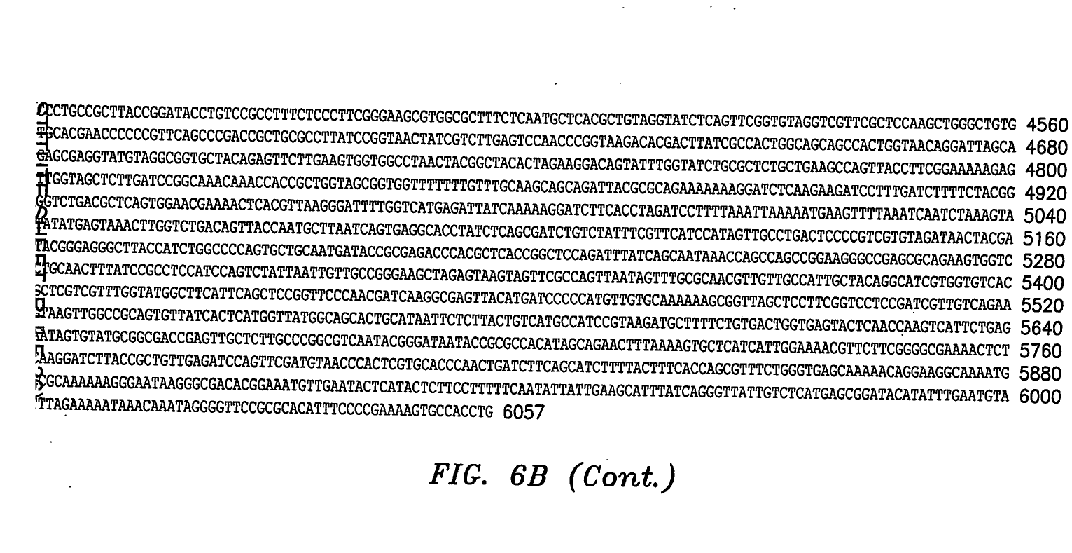

The above procedure was also performed with a pBR322 bacterial plasmid which carried genomic DNA encoding the human IgG2 CH1, hinge region and first part of CH2, which were excised with PmllI and Bst EII and subcloned into APEX-1 3F4 VH HuGamma 4 to replace the corresponding IgG4-derived sequences. The sequence of the resulting chimeric IgG2/IgG4 human constant region is shown in FIG. 6 (APEX-1 3F4 VH G2/G4).

Construction of Modified G2G4 Constant Region

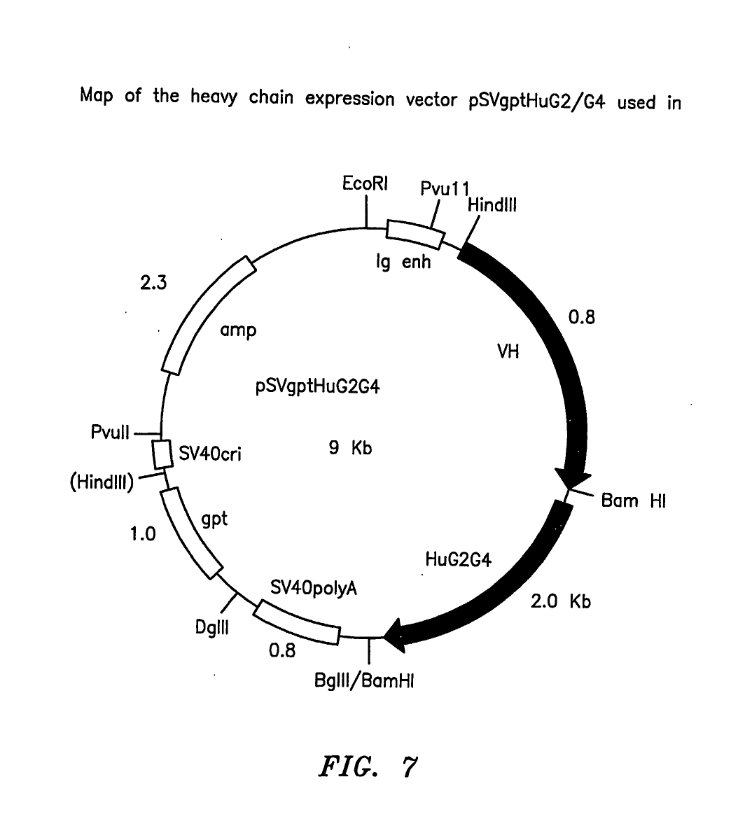

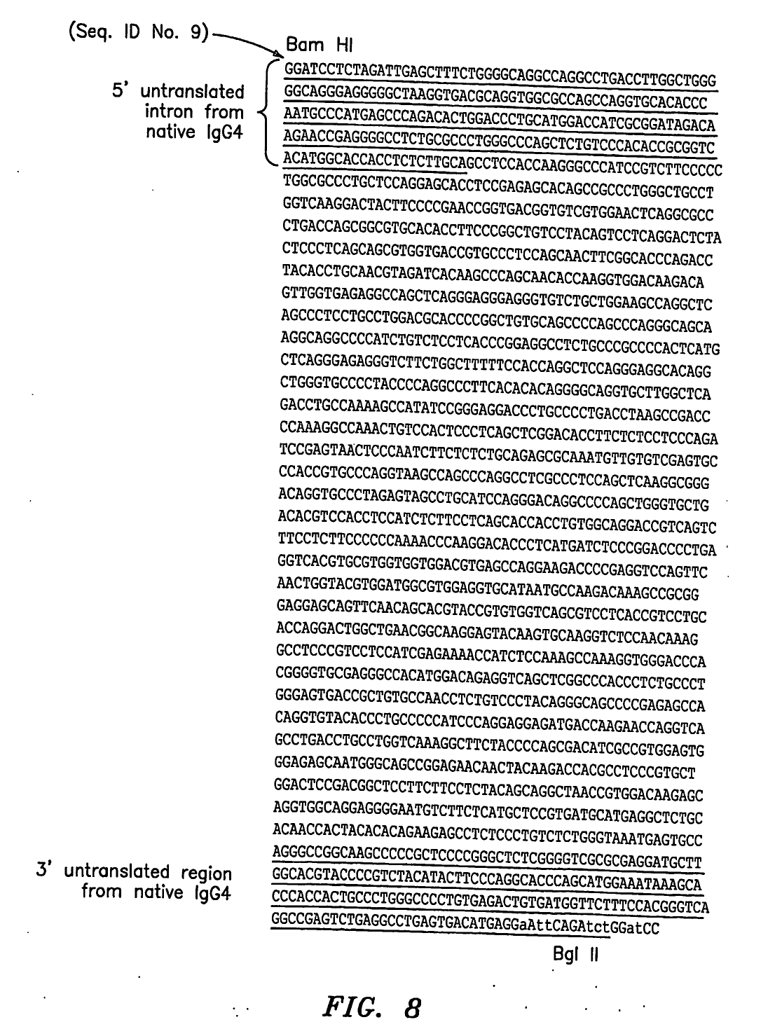

The HuG2G4 constant region was modified for insertion into a heavy chain expression vector as follows: The 5′ end of the HuIgG4 constant region (native HuIgG4 5′ intron sequence with BamH1 site at 5′ end) up to the start of the coding region is amplified in reaction 1. The Hu G2G4 coding sequence (including intron) is amplified from APEX-1 3F4 VH Hu G2/G4 vector in reaction 2 (from start of CH1 to end of CH3 region). The 3′ end of native HuIgG4 3′ sequence from the end of the CH3 coding region is amplified in reaction 3, using a 3′ primer designed to introduce a 3′ Bam HI site with a Bgl II site just inside the Bam H1 and an Eco R1 site just inside the Bgl II site. The products of these 3 reactions (which overlap) are combined in a 4^{th} PCR reaction using 5′ and 3′ primers. The combined product is cloned into the BamH1 site of pUC19 and the DNA sequence of the modified HuG2G4 fragment confirmed. The G2G4 gene is cut out with Bgl II and Bam HI to give a fragment with Bam HI at the 5′ end and Bgl II at the 3′ end. This is cloned into a heavy chain expression vector cut with Bam HI. A clone with the constant region inserted in the correct orientation (Bam HI site reformed at 5′ end, hybrid Bam HI/BglII site at 3′ end) is selected (FIG. 7). The complete sequence of the Bam HI to Bgl II fragment is shown in FIG. 8. Antibody Variable regions can be cloned in directly as Hind III to Bam HI fragments.

| | |
|---|---|
| Primers (SEQ ID NOS: 77-83): | |
| TTGTGAGCGGATAACAATTTC | M13 −50 |
| | REVERSE |
| GTTTTCCCAGTCACGACGTTGTA | M13 −40 |
| | FORWARD |
| CTTGCAGCCTCCACCAAGGGCCCATCCGTC | G2G4-1 |
| CCCTTGGTGGAGGCTGCAAGAGAGG | G2G4-2 |
| GAGCCTCTCCCTGTCTCTGGGTAAATGAGTGCC | G2G4-3 |
| TCATTTACCCAGAGACAGGGAGAGGCTCTTCTGTG | G2G4-4 |
| TACCCGGGGATCCAGATCTGAATTCCTCATGTCAC | G2G4-6 |

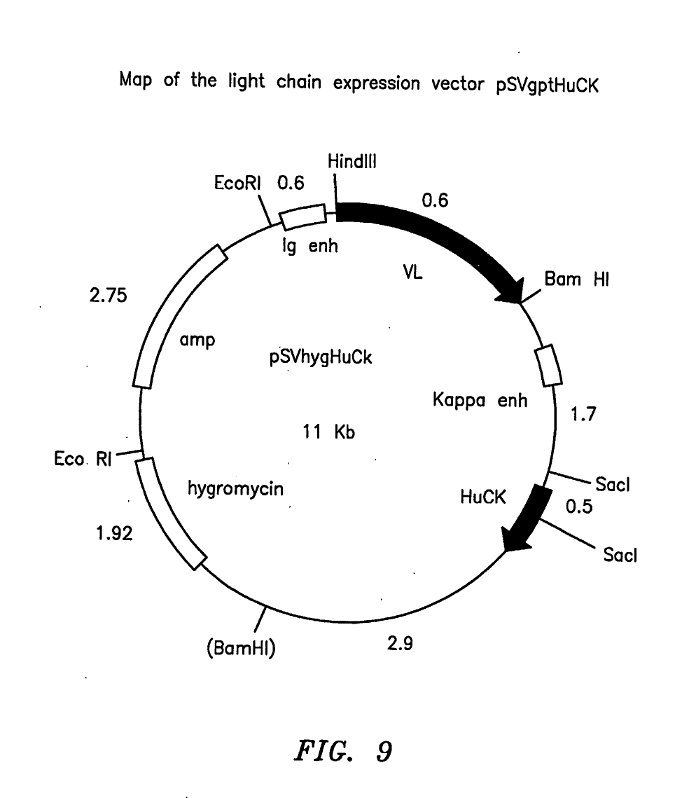

The light chain constant region was the human kappa constant region. This is included in the expression vector pSV hyg HuCκ as shown in FIG. 9.

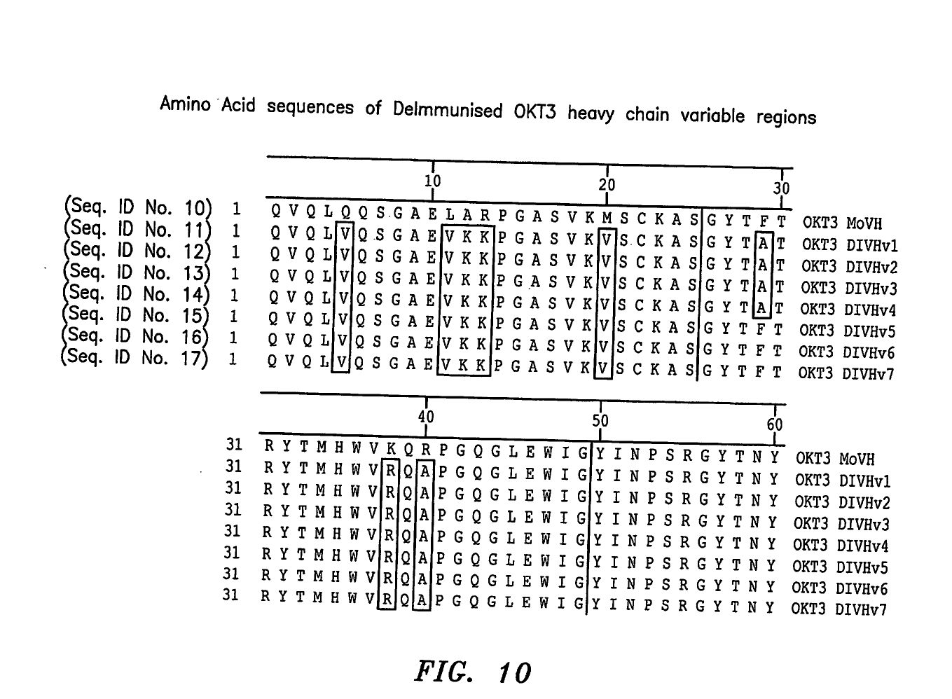

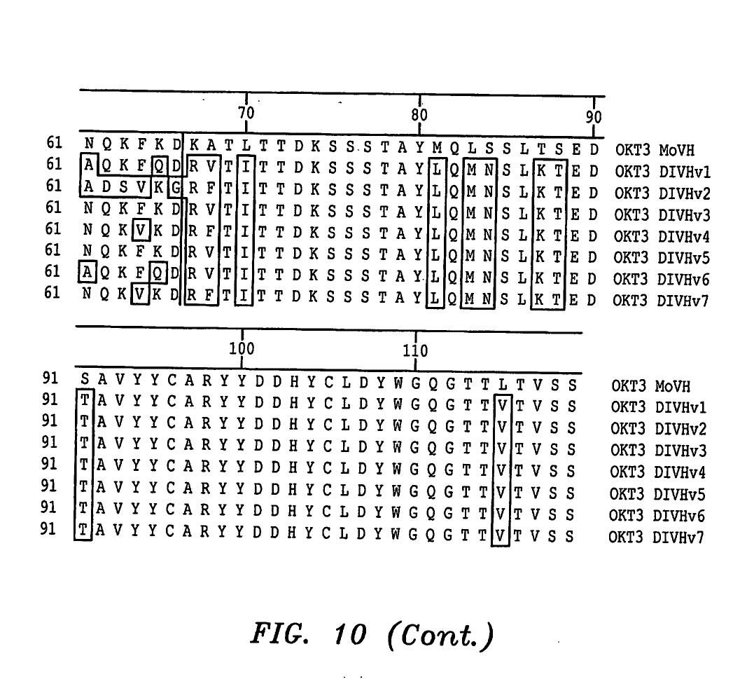

The amino acid sequence of the variable regions of the murine anti-CD3 antibody OKT3 were analyzed for potential T cell epitopes (MHC Class II binding peptides) by using the peptide threading software as detailed in WO 02/069232 published Sep. 6, 2002 and other in silico techniques. De-immunized sequences were designed to eliminate the potential T cell epitopes, as far as possible by making conservative amino acid changes. In order to test the effect on antibody binding of alternative substitutions designed to remove T cell epitopes, several versions of the de-immunized heavy and light chain variable region were constructed, as shown in FIGS. 10 and 11.

The murine OKT3 heavy and light chain variable region cassettes were used as templates for construction of the designed de-Immunized sequences by mutagenesis using overlapping PCR with mutagenic oligonucleotides primers (see FIG. 12 The vectors VH-PCR1 and VK-PCR1 (Riechmann et al., 1988) were used as templates to introduce 5′ flanking sequences including the leader signal peptide sequence, the leader intron and the murine immunoglobulin promoter, and 3′ flanking sequence including the splice site and intron sequences. Sets of mutagenic primer pairs were synthesized encompassing the regions to be altered, such that the target DNA sequence is amplified as a set of fragments.

Adjacent oligos were designed so that the sequences overlap by at least 15 bp. The number of these depends on the number of sites to be mutated.

PCR amplifications for each primer pair were set up using the following reagents:
1 μL template DNA
1 μL (25 pmol) forward primer
1 μL (25 pmol) reverse primer
1 μL 10 mM dNTPs
5 μL 10×Pfu polymerase buffer
0.5 μL (1 unit) Pfu DNA polymerase
H₂O to 50 μL

All reagents except the enzyme were mixed in a 0.5 ml thin wall PCR tube and heated to 94° C. on the PCR block. The Pfu enzyme was added then samples cycled: 94° C./2min, 15-20 cycles of 94° C./30 s, 50° C./30 s, 75° C./1 min (depending on the length of extension required), finishing with 75° C. 5 min. The annealing temperature may be lower or higher than 50° C. depending on the Tm of the oligos.

5 μL of each reaction were run on an agarose gel to check that the PCRs had given products of the expected size. If not, I the annealing temperature was lowered by 5° C. and/or the number of cycles of PCR was increased and/or the MgCl₂ concentration was increased to 5 mM. If a primary PCR gave multiple bands, the band of the correct size was gel-purified.

The products were joined in a second PCR using the 2^{nd} round 5′ and 3′ primers only. This sequence was not present in the original template so only mutagenised DNA can be amplified at this stage. The templates for the 2^{nd} joining PCR were the fragments produced in the first round. The quantities of these were adjusted to add approximately equal amounts. The reagents for the 2^{nd} round PCR were:
Products of 1^{st} round PCR
2 μL (50 pmol) 5′ 2^{nd} round primer
2 μL (50 pmol) 3′ 2^{nd} round primer
1 μL 10 mM dNTPs
5 μL 10×Pfu polymerase buffer
0.5 μL (1 unit) Pfu DNA polymerase
H₂O to 50 μL

All reagents except the enzyme were mixed in a 0.5 ml thin wall PCR tube and heated to 94° C. on the PCR block. The Pfu enzyme was added then samples cycled: 94° C./2min, 15 cycles of 94° C./30 s, 50° C./30 s, 75° C./1 min (depending on the length of extension required), finishing with 75° C. 5 min.

5 μL of each reaction was run on an agarose gel to check that the PCRs had given products of the expected size (approximately 820 bp for VH expression cassettes and 650 bp for VK expression cassettes). If not, the 2^{nd} round PCR was repeated lowering the annealing temperature by 5° C. and/or increasing the number of cycles of PCR.

The remainder of the PCR product was phenol/chloroform extracted and ethanol precipitated, digested with the required enzymes (usually Hind111 and BamH1 for expression cassettes) and loaded onto a 1.5% low-melting point agarose gel. DNA bands of the correct size were excised and purified.

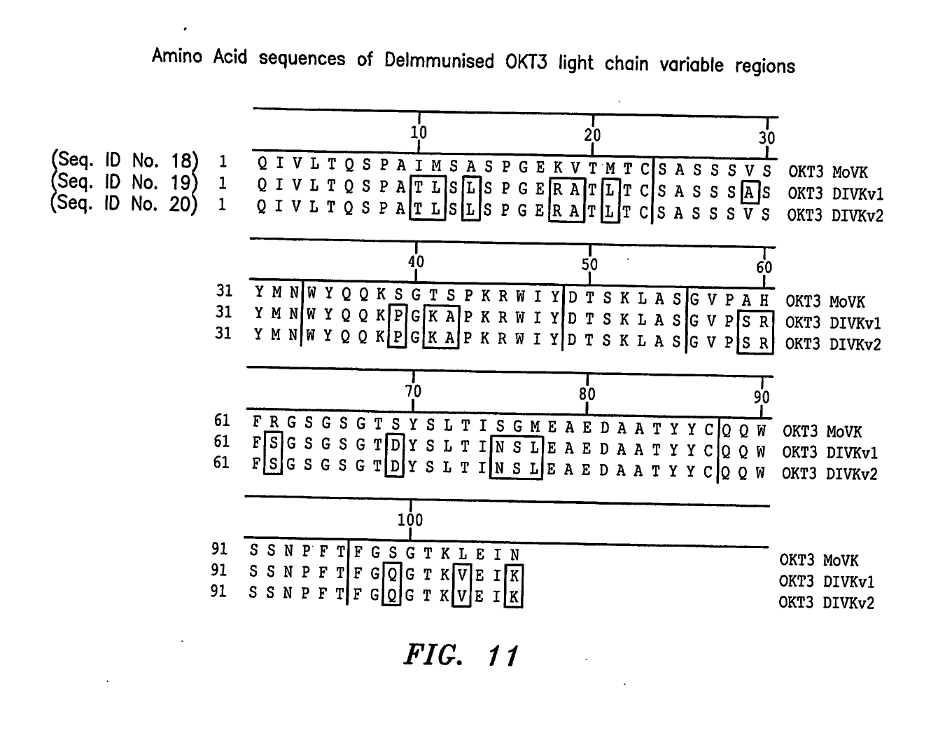

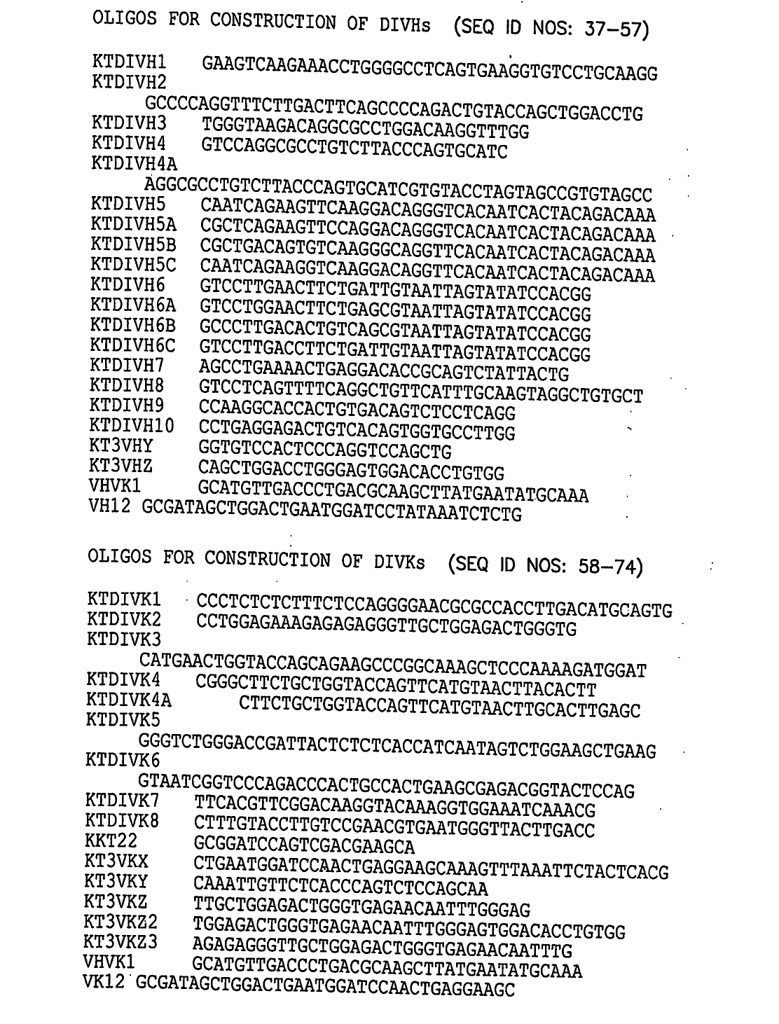

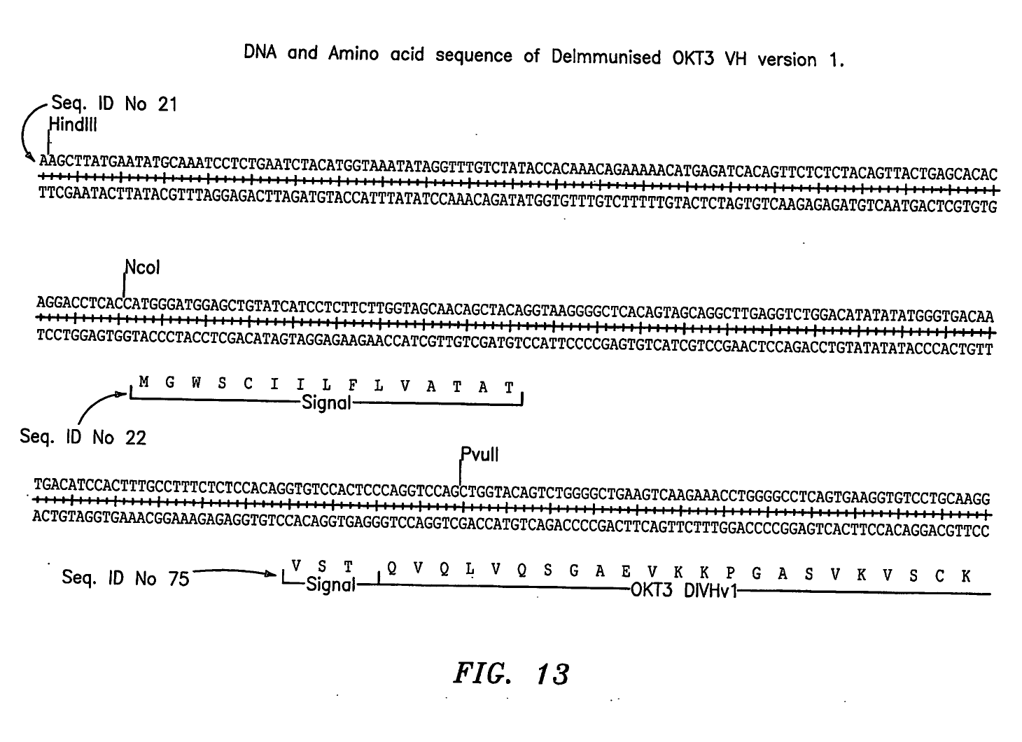

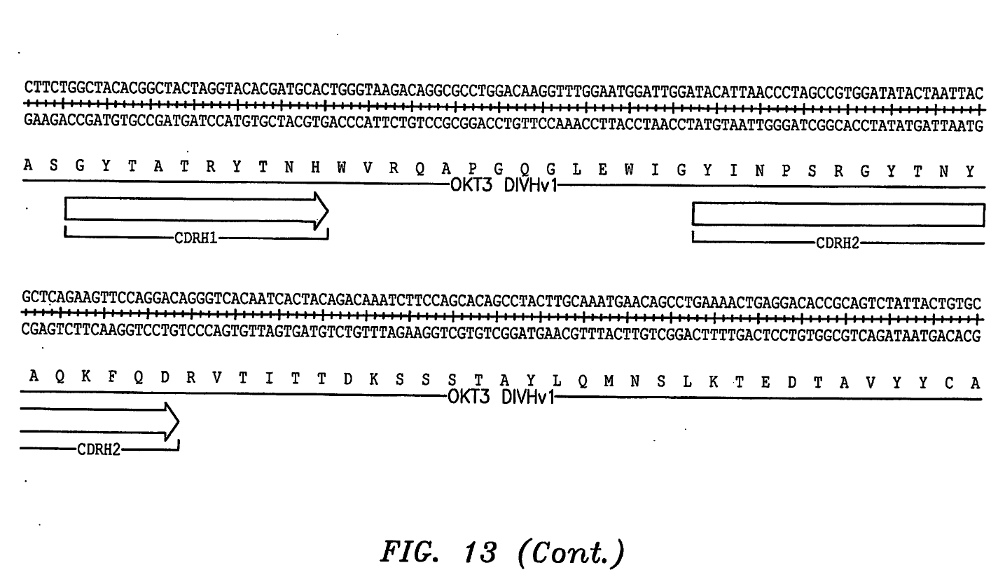

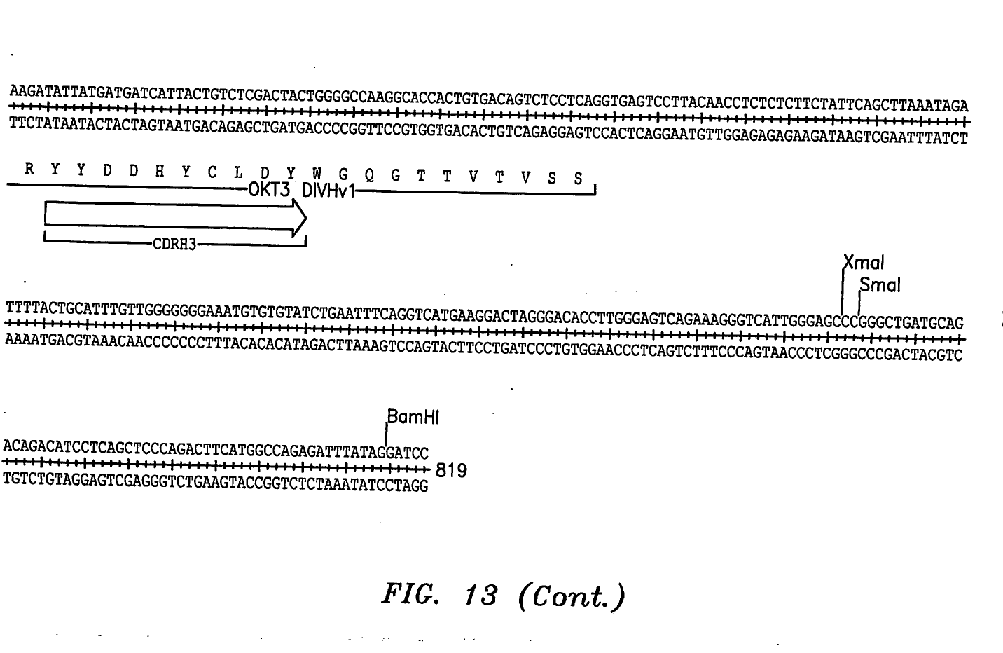

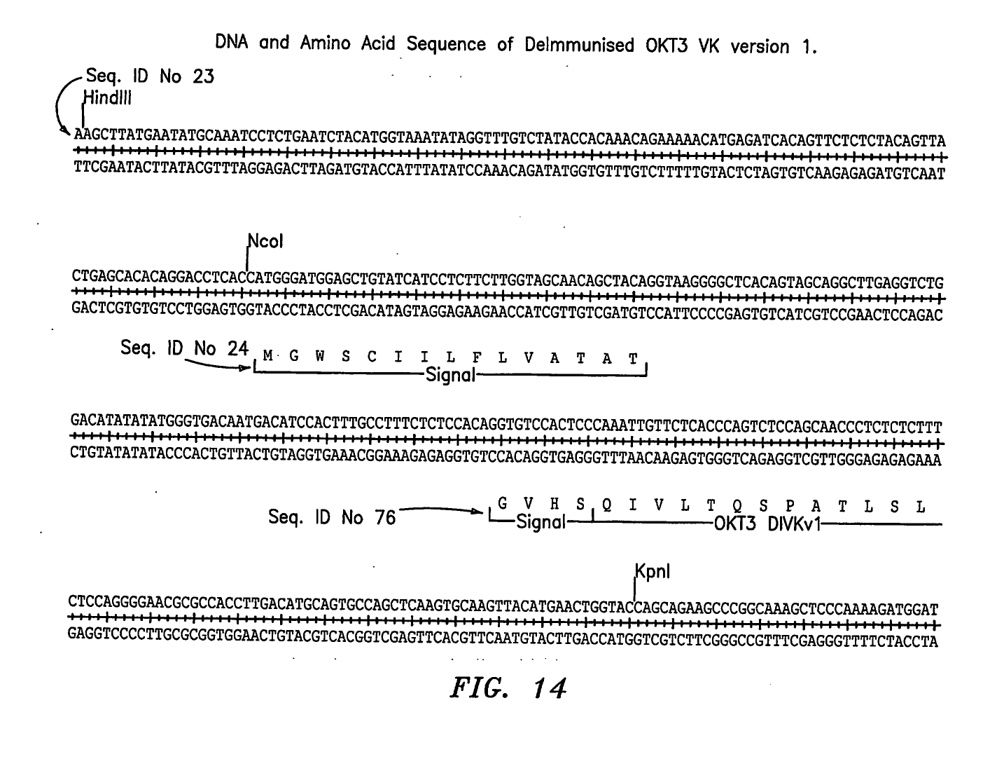

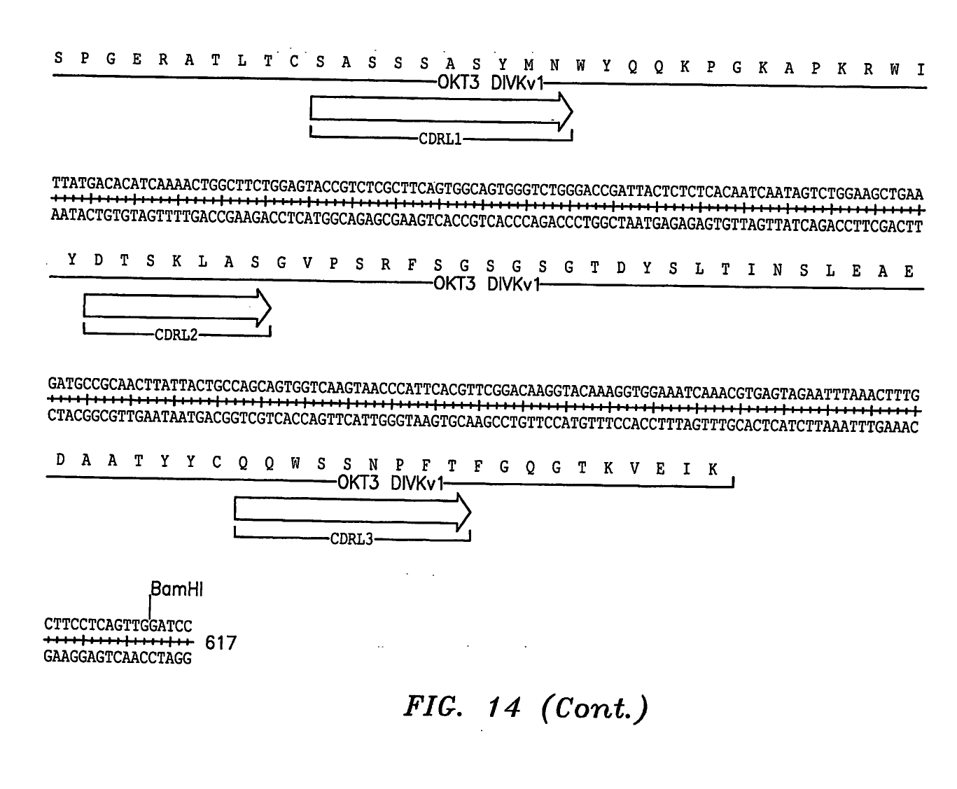

The de-immunized VH and Vκ expression cassettes produced (FIG. 2) were cloned into the vector pUC19 and the entire DNA sequence was confirmed to be correct for each de-immunized VH and Vκ. As an example, the DNA and amino acid sequences for the de-immunized OKT3 VH and Vk 1 expression cassettes OKT3 DIV H V1 (version 1) and OKT3 DIVK V1′ (version 1) are shown in FIGS. 13 and 14, respectively.

The de-immunized heavy and light chain V-region genes were excised from the vector pUC19 as HindIII to BamHI expression cassettes. These were transferred to the expression vectors pSVgpt HuG2G4 and pSVhyg HuCκ (FIGS. 7 and 9, respectively), which include the previously described HuG2G4 or human κ constant regions, respectively, and markers for selection in mammalian cells. The DNA sequence was confirmed to be correct for the de-immunized VH and Vκ in the expression vectors.

The original murine OKT3 heavy and light chain light chain variable region cassettes were also transferred to the expression vectors pSVgpt HuG2G4 and pSVhyg HuCκ as described above, to generate a chimeric antibody with the murine variable region genes linked to the human constant region G2/G4 construct. This chimeric antibody was used as an isotype matched control for binding experiments with the de-immunized antibodies, as it has with the same effector functions and the same secondary detection reagents are used as for the de-immunized antibodies.

The host cell line for antibody expression was NSO, a non-immunoglobulin producing mouse myeloma, obtained from the European Collection of Animal Cell Cultures, Porton UK (ECACC No 85110503). The heavy and light chain expression vectors were co-transfected into NSO cells by electroporation. The transfection was accomplished as follows: DNA to be transfected was linearized to improve efficiency. PvuI digests of about 3 and 6 mg of the plasmids pSVgpt HuG2/G4 and pSV hyg HUCK, respectively, were prepared. The digested DNA was ethanol precipitated and dissolved in 50 ml dH₂O. Recipient NSO cells were resuspended from a semi-confluent 75 cm² flask and collected by centrifugation at 1000 rpm for 5 min. The supernatant was discarded. The cells were resuspended in 0.5 ml DMEM and transferred to a Gene Pulser cuvette (Bio-Rad). The DNA was mixed with the cells by gentle pipetting and left on ice for 5 minutes. The cuvette was inserted between the electrodes of the Bio-rad Gene Pulser and a single pulse of 170 V, 960 mF was applied. The cuvette was then returned to ice for 20 minutes. The cell suspension was transferred to a 75 cm² flask containing 20 ml DMEM and allowed to recover for 1-2 days. Cells were harvested and resuspended in 80 ml selective DMEM and a 200 μL aliquot was added to each well of 96-well plates. Selective DMEM is Dulbecco's Modified Eagle's Medium (DMEM) supplemented with 10% (v/v) foetal calf serum, 250 μg/ml xanthine, 0.8 μg/ml mycophenolic acid. Approximately 10 days from the start of selection, colonies were visible to the naked eye. 20 μL of medium from each well was assayed for the presence of human antibodies. On the basis of the level of antibody production and the number of cells in the well, wells were chosen for expansion. To resuspend the cells from the designated wells, the tip of a Gilson P200 pipette (with yellow tip) was rubbed across the surface and the medium transferred to a well of a 24-well tissue culture plate containing 1.5 ml of fresh selective DMEM. Cells were expanded to 25 cm² and larger tissue culture flasks in order to lay down liquid nitrogen stocks and to provide medium for antibody purification and testing.

Each of the 7 de-immunized heavy chain genes (FIG. 10) was paired with each of 2 de-immunized light chain genes (FIG. 11) to give a total of 14 de-immunized OKT3 antibodies to be produced. The chimeric heavy and light chain vectors were co-transfected to produce the chimeric antibody. Colonies expressing the gpt gene were selected using selective DMEM. Transfected cell clones were screened for production of human antibody by ELISA for human IgG as follows: An ELISA plate (Dynatech Immulon 2) was coated at 100 μL per well with sheep anti-human κ antibody (The Binding Site Cat No: AU015) diluted 1:1000 in carbonate/bicarbonate coating buffer pH9.6 (Sigma Cat: C-3041). The samples were incubated at 4° C. overnight. After washing 3 times with a solution of PBS with 0.05% Tween 20 (RTM), Where transfections were plated into 96-well plates, screening was conducted using 25 μL samples of culture medium from each well by transfer into an assay plate containing 75 μL per well of PBS/Tween (PBST) solution. Where transfected cells were cultured using 24-well plates, 12.5 μL was added to 87.5 μL in the first well and a doubling dilution series set out across the plate. For all assays, blank wells received PBST only. The samples were incubated at room temperature for 1 hour and washed 3 times with the PBS/Tween solution. The secondary antibody, a peroxidase-conjugated sheep anti-human IgG γ chain specific reagent (The Binding Site Cat No: AP004), was added at a ratio of 1:1000 in the PBS/Tween solution in an amount of 100 μL per well. The samples were again incubated at room temperature for 1 hour and washed 3 times with the PBS/Tween solution. To prepare the colour substrate, one tablet (20mg) of OPD (o-PHENYLENE DIAMINE) (Sigma Cat No: P-7288) was dissolved in 45 ml of H₂O plus 5 ml 10× peroxidase buffer (make 10× peroxidase buffer with Sigma phosphate citrate buffer tablets pH 5.0, Cat No: P-4809), and 10 mL 30% (w/w) hydrogen peroxide (Sigma Cat No: H-1109) was added just before use. The substrate was added at 100 μL per well and incubated at room temperature for 5 min. or as required. When colour developed, the process was stopped by adding 25 μL 12.5% H₂SO₄. The result was read at 492 nm. The standard antibody employed was Human IgG1/κ purified myeloma protein (The Binding Site Cat No: BP078).

Cell lines secreting antibody were expanded and the highest produces selected. De-immunized and chimeric antibodies were purified using Prosep®-A (Bioprocessing Ltd, Durham, UK) as follows: The NSO transfectoma cell line producing antibody was grown in DMEM 5% FCS in Nunc Triple layer flasks, 250 ml per flask (total volume 1 L) for 10-14 days until nearing saturation. The conditioned medium was collected and spun at 3000 rpm for 5 minutes in a bench centrifuge to remove cells. One tenth the volume of 1M Tris-HCL pH8 (Sigma Cat: T3038) was added to the cell supernatant to make this 0.1M Tris-HCL pH8. 0.5 to ml of Prosep A (Millipore Cat: 113111824) was added and stirred overnight at room temperature. Prosep A was collected by spinning at 3000 rpm for 5 minutes then packed into a Biorad Poly-Prep column (Cat: 731-1550). The column was washed with 10 ml PBS, then eluted in 1 ml fractions with 0.1 M Glycine pH3.0. Each fraction was collected into a tube containing 100 μL 1 M Tris-HCL pH8 (Sigma, as above). The absorbance of each fraction measured at 280 nm. The fractions containing antibody were pooled and dialysed against PBS overnight at room temperature. The preparation was sterilized by filtration through a 0.2 micron syringe filter and the A₂₈₀ was measured. The concentration was determined by ELISA for human IgG1 κ.

Evaluation of Binding of De-immunized Antibodies to Cells Expressing CD3

Each de-immunized antibody was evaluated for its ability to bind the CD3 molecule on T cells as it was possible that the mutations introduced during de-immunization could affect antibody specificity or affinity. Cells of the HPB-ALL (Human peripheral blood acute lymphocytic leukemia) line were obtained from the Cell Resource Center for Biomedical Research, Tohoku University, Japan. Jurkat and J.RT3 cell lines were obtained from American Type Tissue Culture (ATCC), Rockville, Md. Cells were cultured at 2×10⁵-2×10⁶ cells/ml in RPMI 1640 (Cellgro) containing 10% heat-activated fetal bovine serum (Atlas Biologicals), 1% penicillin-streptamycin, 0.01M Hepes (Sigma), 0.2 mM 1-glutamine and 5×10⁻⁵M 2-mercaptoethanol (Sigma) at 37° C. in a humidified chamber containing 5% CO₂ in air. HPB-ALL and Jurkat cells bear high levels of the TCR/CD3 complex on their cell surface, while the J.RT3 cells are a variant of the Jurkat line that do not express CD3. Preparations of the murine OKT3, chimeric OKT3 and de-immunized OKT3 G2/G4 antibodies were evaluated for binding to cells of all three lines by immunocytochemistry and flow cytometry. Briefly, 10⁶ cells were plated in individual wells of a 96-well plate and reacted with 1 μg of each test antibody or with appropriate human or mouse isotype controls for 20 minutes at 4° C. The cells were then washed three times with PBS containing 2% fetal bovine serum (Atlas Biologicals) followed by reactivity for 20 minutes at 4° C. with a phycoerthrin-conjugated secondary antibody (R-PE Affinity pure F(ab)2 goat anti-human IgG (H+L) (Jackson ImmunoResearch, Bar Harbor, Me.) for the detection of the chimeric and de-immunized antibodies and G2/G4 isotype control, and R-PE-conjugated goat anti-mouse IgG (Pharmingen), for the detection of the murine OKT3 and murine IgG2a isotype control). The cells were washed three times with PBS-FBS as above, and then resuspended in PBS for analysis on a flow cytometer (FACs Calibur, Becton Dickenson).

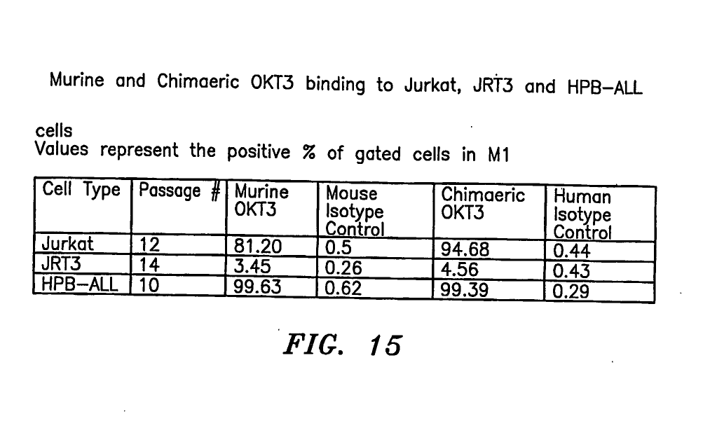

Both the chimeric OKT3 HuG2/G4κ antibody and the murine OKT3 antibody bound comparably to Jurkat and HPB-AII cells that express CD3, but showed no binding to the CD3 negative cell line J.RT3. The matched murine and human isotype controls showed no binding to any of the cell types (FIG. 15)

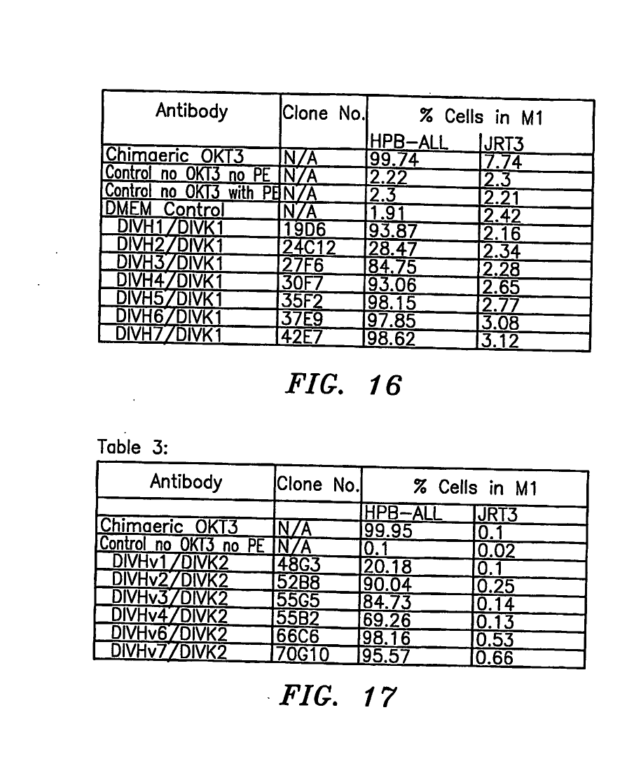

Conditioned media from NSO cell lines expressing de-immunised OKT3 antibodies were also tested in the flow cytometry binding assay using HPB-ALL and J.RT3 cells. The concentration of antibody in the conditioned medium was determined by ELISA for human IgG using the ELISA procedure previously described. The immunocytochemistry and flow cytometry procedures were performed as described above. The results for the 7 versions of de-immunised heavy chain combined with de-immunised OKT3 light chain version 1 are shown in FIG. 16. The results for the de-immunised heavy chains combined with de-immunised OKT3 light chain version 2 are shown in FIG. 17. A number of the de-immunised OKT3 antibodies demonstrated binding to HPB-ALL cells equivalent to that observed for murine and chimeric antibodies. However, several of the de-immunized antibodies showed a significantly lower level of binding to HPB-ALL cells (e.g. antibodies derived from clones 24C12, 48G3, and 55B2). The ability of a given antibody to bind to HPB-ALL cells did not correlate with the usage of a particular version of kappa light chain; i.e., both mutated kappa chains, in combination with some, but not all of the mutated VH regions, showed comparable binding in this assay.

Competition Assay Comparing Binding of Murine, Chimeric and De-Immunized OKT3 Antibodies

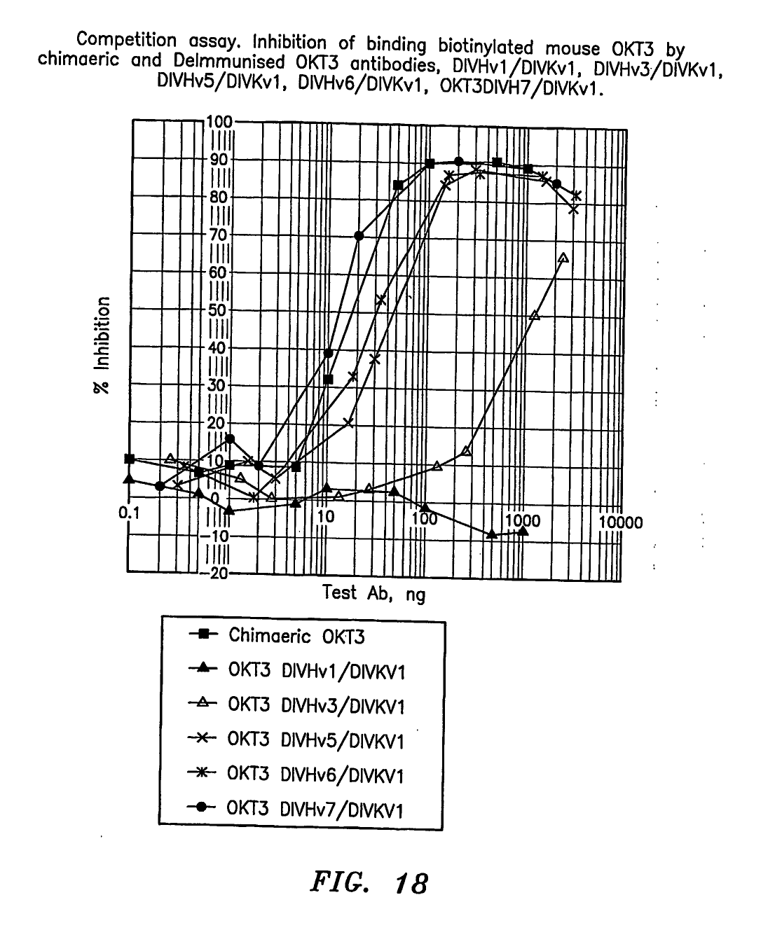

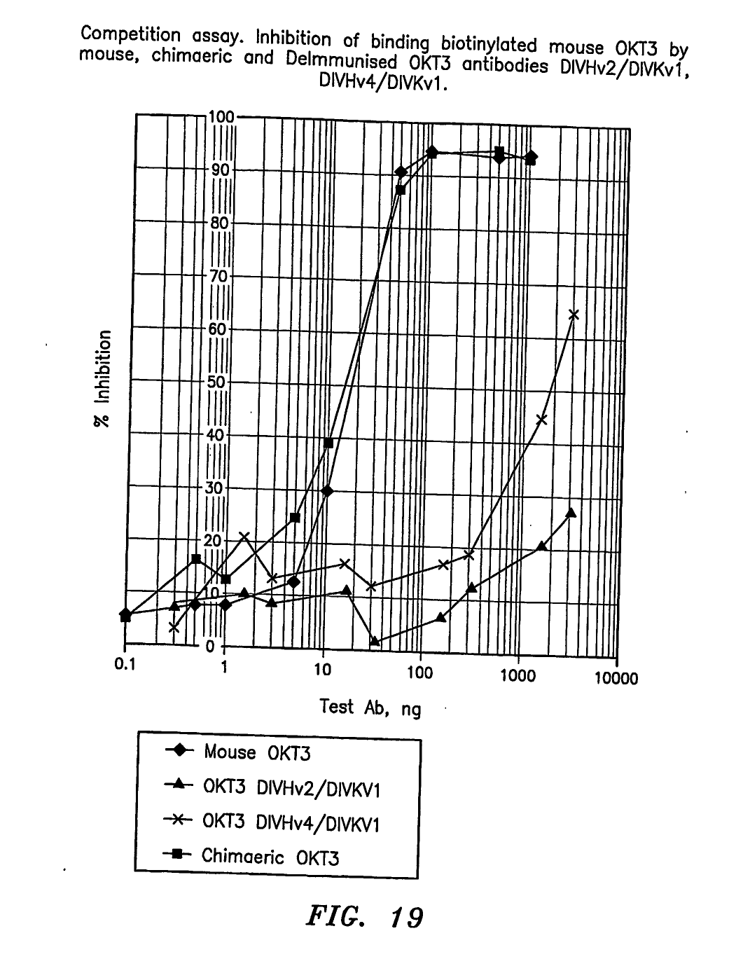

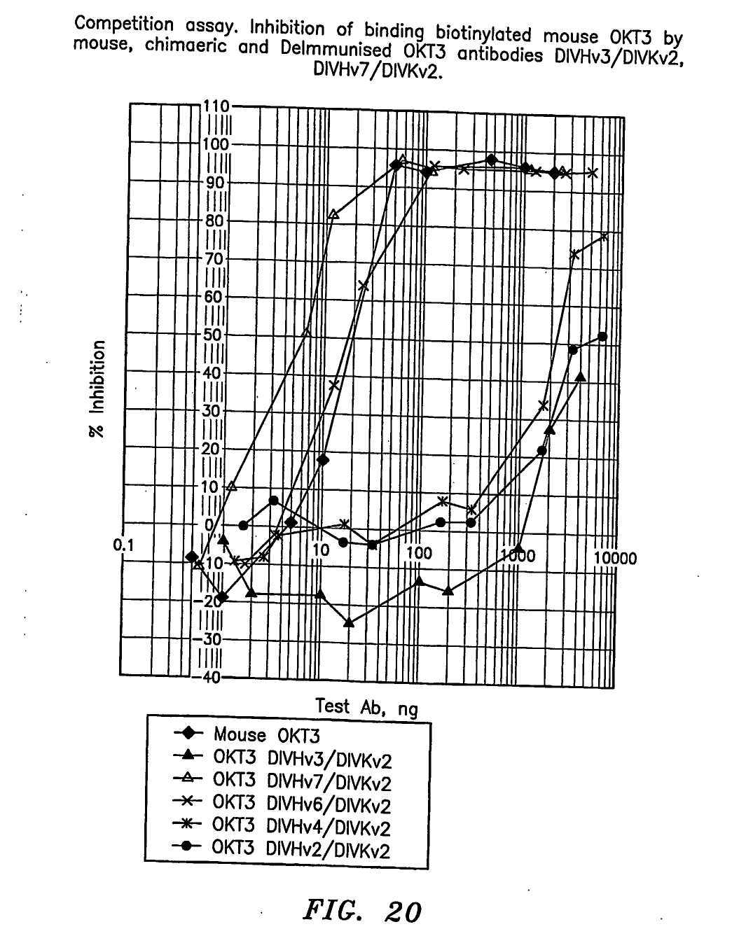

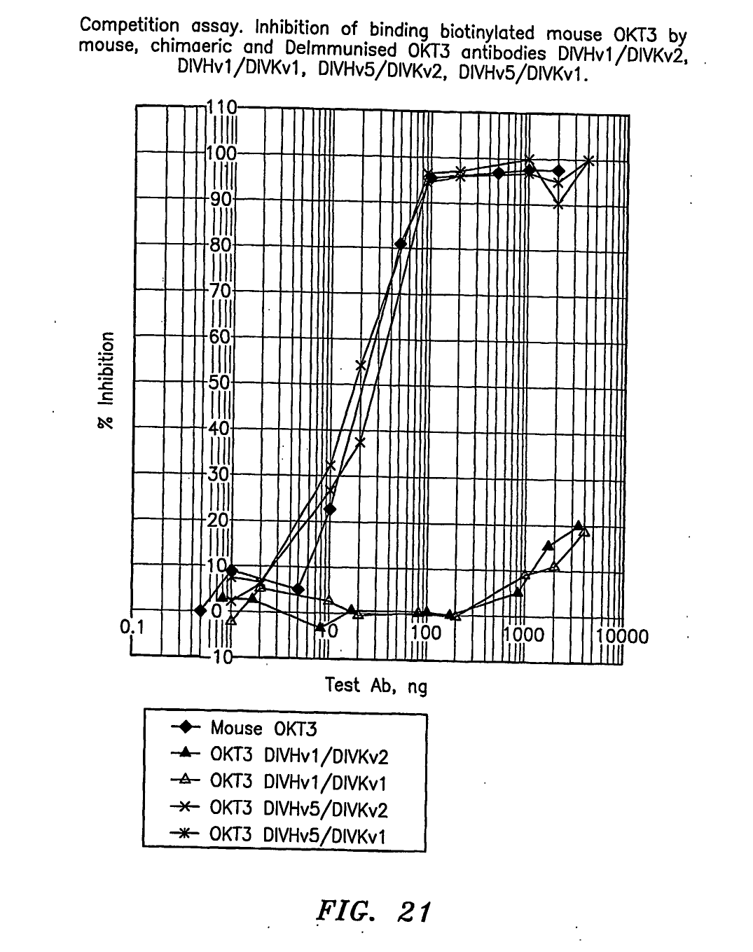

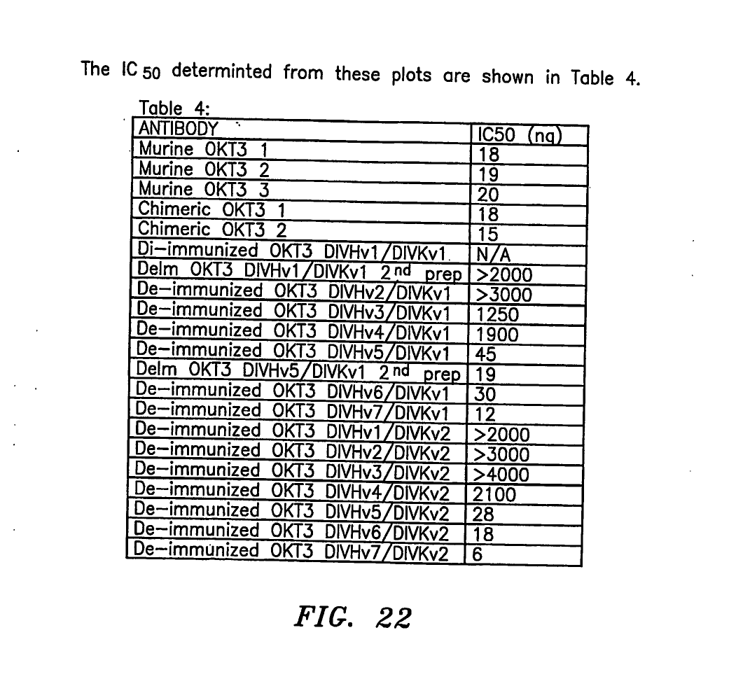

To obtain more discrimination between the various de-immunized antibodies, and to determine their binding affinity relative to that of OKT3, a competition binding assay was carried out. Because CD3 is part of a cell-surface complex of proteins, affinities could not be measured by BIACORE analysis, but instead were measured by flow cytometry. Murine OKT3 was biotinylated using EZ-Link Sulfo-NHS-LC Biotin from Perbio Science, catalogue number 21335, following the protocol provided by the manufacturer. The amount of biotinylated OKT3 to use was determined by titrating HPB-ALL cells with decreasing amounts of antibody. A suitable sub-saturating concentration was determined to be 10 ng of biotinylated antibody per 10⁶ cells. This concentration was then used in all experiments. The secondary detection reagent was avidin-FITC, Sigma catalogue number A2050. Competition with dilutions of test (de-immunized or chimeric) antibodies from 100 pg to 1 μg was tested. The results are expressed as percent inhibition of maximal fluorescence activity (determined by binding of biotinylated murine OKT3 in the absence of blocking antibody) and are shown in FIGS. 18, 19, 20 and 21.

The results show that the chimeric OKT3 antibody and the six de-immunized antibodies OKT3 DIVHv5 to DIVGv7/DIVKv1 and OKT3 DIVH5 to DIVH7/DIVK2 can compete with the binding of the biotinylated murine OKT3 antibody either as efficiently as the murine antibody itself or within two to three fold of it.

Throughout this specification, various publications and patent disclosures are referred to. The teachings and disclosures thereof, in their entireties, are hereby incorporated by reference into this application to more fully describe the state of the art to which the present invention pertains.

Although preferred and other embodiments of the invention have been described herein, further embodiments may be perceived by those skilled in the art without departing from the scope of the invention as defined by the following claims.

1. A de-immunized anti-CD3 antibody.

2. A de-immunized anti-CD3 antibody heavy chain variable region comprising a sequence selected from the group consisting of SEQ. ID NOS: 11, 12, 13, 14, 15, 16 and 17.

3. A de-immunized anti-CD3 antibody light chain variable region comprising a sequence selected from the group consisting of SEQ. ID NOS: 19 and 20.

4. A method comprising: selecting an anti-CD3 antibody; and rendering the anti-CD3 antibody less immunogenic to a given species.

5. A method as in claim 4 wherein the step of rendering the anti-CD3 antibody less immunogenic to a given species comprises the steps of:
a) determining at least part of the amino acid sequence of the antibody;
(b) identifying in the amino acid sequence one or more potential epitopes for T cells (“T cell epitopes”) which are found in an endogenous protein of the given species; and
(c) modifying the amino acid sequence to eliminate at least one of the T cell epitopes identified in step (b) thereby to reduce the immunogenicity of the antibody or part thereof when exposed to the immune system of the given species.

6. A method comprising the steps of:
(a) producing an expression vector having a DNA sequence which includes a sequence that encodes an anti-CD3 antibody, at least a portion of which has been a deimmunized;
(b) transfecting a host cell with the vector; and
(c) culturing the transfected cell line to produce a de-immunized anti-CD3 antibody molecule.

7. A pharmaceutical composition comprising a de-immunized anti-CD3 antibody and a pharmaceutical acceptable carrier.

8. A composition as in claim 7 wherein the de-immunized anti-CD3 antibody includes a heavy chain variable region comprising a sequence selected from the group consisting of SEQ. ID NOS: 11, 12, 13, 14, 15, 16 and 17.

9. A composition as in claim 7 wherein the de-immunized anti-CD3 antibody includes a light chain variable region comprising a sequence selected from the group consisting of SEQ. ID NOS: 19 and 20.

10. A method comprising administering an anti-CD3 antibody, the anti-CD3 antibody including an engineered heavy chain constant region having a first portion derived from one or more humanIgG2 antibodies and a second portion derived from one or more humanIgG4 antibodies, at least a portion of the antibody being deimmunized.

11. A method as in claim 10 wherein at least the light chain variable region of the antibody is de-immunized.

12. A method as in claim 10 wherein at least the heavy chain variable region of the antibody is de-immunized.

13. A method as in claim 10 wherein both the light and heavy chain variable regions of the antibody are de-immunized.

14. A method as in claim 10 wherein the anti-CD3 antibody includes a heavy chain variable region comprising a sequence selected from the group consisting of SEQ. ID NOS: 11, 12, 13, 14, 15, 16 and 17.

15. A method as in claim 10 wherein the anti-CD3 antibody includes a light chain variable region comprising a sequence selected from the group consisting of SEQ. ID NOS: 19 and 20.

16. A method as in claim 10 wherein at least a portion of the antibody is deimmunized by a process comprising the steps of: rendering the antibody, or part thereof, non-immunogenic, or less immunogenic, to a given species by
a) determining at least part of the amino acid sequence of the antibody;
(b) identifying in the amino acid sequence one or more potential epitopes for T cells (“T cell epitopes”) which are found in an endogenous protein of the given species; and
(c) modifying the amino acid sequence to eliminate at least one of the T cell epitopes identified in step (b) thereby to reduce the immunogenicity of the antibody or part thereof when exposed to the immune system of the given species.

17. Nucleic acid encoding a de-immunized anti-CD3 antibody.

18. Nucleic acid in accordance with claim 17 which encodes an antibody heavy chain variable region comprising a sequence selected from the group consisting of SEQ. ID NOS: 11, 12, 13, 14, 15, 16 and 17.

19. Nucleic acid in accordance with claim 17 which encodes an antibody light chain variable region comprising a sequence selected from the group consisting of SEQ. ID NOS: 19 and 20.

20. A pharmaceutical composition comprising an anti-CD3 antibody encoded by the nucleic acid in accordance with claim 17 and a pharmaceutical acceptable carrier.


SEQUENCE LISTING (83 sequences)

SEQ ID NO: 1 - DNA, 819 bp, murine
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactcccaggtccagctgcaacagtctggggctgaactcgcaagacctggggcctcagtgaagatgtcctgcaaggcttctggctacacgtttactaggtacacgatgcactgggtaaaacagaggcctggacaaggtttggaatggattggatacattaaccctagccgtggatatactaattacaatcagaagttcaaggacaaggccacactgactacagacaaatcttccagcacagcctacatgcaactgagcagcctgacatctgaggactccgcagtctattactgtgcaagatattatgatgatcattactgtctcgactactggggccaaggcaccactttgacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagcccgggctgatgcagacagacatcctcagctcccagacttcatggccagagatttataggatcc

SEQ ID NO: 2 - protein, 15 aa, murine
MGWSCIILFLVATAT

SEQ ID NO: 3 - DNA, 617 bp, murine
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactcccaaattgttctcacccagtctccagcaatcatgtctgcatctccaggggaaaaggtcaccatgacatgcagtgccagctcaagtgtaagttacatgaactggtaccagcagaagtcaggcacctcccccaaaagatggatttatgacacatcaaaactggcttctggagtaccggctcacttcaggggcagtgggtctgggacctcttactctctcacaatctcagggatggaagctgaagatgccgcaacttattactgccagcagtggtcaagtaacccattcacgttcggatctggtacaaagttggaaatcaaacgtgagtagaatttaaactttgcttcctcagttggatcc

SEQ ID NO: 4 - protein, 15 aa, murine
MGWSCIILFLVATAT

SEQ ID NO: 5 - DNA, 6058 bp, artificial sequence
  vector
acgcgttgacattgattattgactagttattaatagtaatcaattacggggtcattagttcatagcccatatatggagttccgcgttacataacttacggtaaatggccccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattctgttgggctcgcggttgattacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgaccggcttggtaccgagctcggatccggaccatcatgaagtggagctgggttattctcttcctcctgtcagtaactgccggcgtccactcccaggttcaggtccagcagtctggggctgagctggcaagaccttgggcttcagtgaagttgtcctgcaaggcttctggctacaattttaatagttactggatgcagtgggtaaaacagaggcctggacagggtctggaatggattggggctatttatcctggagatggtgatactagctacactcagaagttcaggggcaaggccacattgactgcagataaatcctccagcacagcctacatgcaactcagcagcttggcatctgaggactctgcggtctattactgtgcaagacgtacggtaggaggctactttgactactggggccaaggcaccactctcacagtctcctcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccatccatcacactggcggccgctcgagcatgcatctagaacttgtttattgcagcttataatggttacaaataaagcaatagcatcacaaatttcacaaataaagcatttttttcactgcattctagttgtggtttgtccaaactcatcaatgtatcttatcatgtctggatcgatcccgccatggtatcaacgccatatttctatttacagtagggacctcttcgttgtgtaggtaccgctgtattcctagggaaatagtagaggcaccttgaactgtctgcatcagccatatagcccccgctgttcgacttacaaacacaggcacagtactgacaaacccatacacctcctctgaaatacccatagttgctagggctgtctccgaactcattacaccctccaaagtcagagctgtaatttcgccatcaagggcagcgagggcttctccagataaaatagcttctgccgagagtcccgtaagggtagacacttcagctaatccctcgatgaggtctactagaatagtcagtgcggctcccattttgaaaattcacttacttgatcagcttcagaagatggcggagggcctccaacacagtaattttcctcccgactcttaaaatagaaaatgtcaagtcagttaagcaggaagtggactaactgacgcagctggccgtgcgacatcctcttttaattagttgctaggcaacgccctccagagggcgtgtggttttgcaagaggaagcaaaagcctctccacccaggcctagaatgtttccacccaatcattactatgacaacagctgttttttttagtattaagcagaggccggggacccctgggcccgcttactctggagaaaaagaagagaggcattgtagaggcttccagaggcaacttgtcaaaacaggagtgcttctatttctgtcacactgtctggccctgtcacaaggtccagcacctccataccccctttaataagcagtttgggaacgggtgcgggtcttactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaaggagctcccagcaaaaggccaggaaccgtaaaaaggccgccttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 6 - protein, 235 aa, human
MKWSWVILFLLSVTAGVHSQVQVQQSGAELARPWASVKLSCKASGYNFNSYWMQWVKQRPGQGLEWIGAIYPGDGDTSYTQKFRGKATLTADKSSSTAYMQLSSLASEDSAVYYCARRTVGGYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 7 - DNA, 6057 bp, artificial sequence
  vector
acgcgttgacattgattattgactagttattaatagtaatcaattacggggtcattagttcatagcccatatatggagttccgcgttacataacttacggtaaatggccccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattctgttgggctcgcggttgattacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgaccggcttggtaccgagctcggatccggaccatcatgaagtggagctgggttattctcttcctcctgtcagtaactgccggcgtccactcccaggttcaggtccagcagtctggggctgagctggcaagaccttgggcttcagtgaagttgtcctgcaaggcttctggctacaattttaatagttactggatgcagtgggtaaaacagaggcctggacagggtctggaatggattggggctatttatcctggagatggtgatactagctacactcagaagttcaggggcaaggccacattgactgcagataaatcctccagcacagcctacatgcaactcagcagcttggcatctgaggactctgcggtctattactgtgcaagacgtacggtaggaggctactttgactactggggccaaggcaccactctcacagtctcctcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccatccatcacactggcggccgctcgagcatgcatctagaacttgtttattgcagcttataatggttacaaataaagcaatagcatcacaaatttcacaaataaagcatttttttcactgcattctagttgtggtttgtccaaactcatcaatgtatcttatcatgtctggatcgatcccgccatggtatcaacgccatatttctatttacagtagggacctcttcgttgtgtaggtaccgctgtattcctagggaaatagtagaggcaccttgaactgtctgcatcagccatatagcccccgctgttcgacttacaaacacaggcacagtactgacaaacccatacacctcctctgaaatacccatagttgctagggctgtctccgaactcattacaccctccaaagtcagagctgtaatttcgccatcaagggcagcgagggcttctccagataaaatagcttctgccgagagtcccgtaagggtagacacttcagctaatccctcgatgaggtctactagaatagtcagtgcggctcccattttgaaaattcacttacttgatcagcttcagaagatggcggagggcctccaacacagtaattttcctcccgactcttaaaatagaaaatgtcaagtcagttaagcaggaagtggactaactgacgcagctggccgtgcgacatcctcttttaattagttgctaggcaacgccctccagagggcgtgtggttttgcaagaggaagcaaaagcctctccacccaggcctagaatgtttccacccaatcattactatgacaacagctgttttttttagtattaagcagaggccggggacccctgggcccgcttactctggagaaaaagaagagaggcattgtagaggcttccagaggcaacttgtcaaaacaggactgcttctatttctgtcacactgtctggccctgtcacaaggtccagcacctccataccccctttaataagcagtttgggaacgggtgcgggtcttactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaaggagctcccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 8 - protein, 235 aa, human
MKWSWVILFLLSVTAGVHSQVQVQQSGAELARPWASVKLSCKASGYNFNSYWMQWVKQRPGQGLEWIGAIYPGDGDTSYTQKFRGKATLTADKSSSTAYMQLSSLASEDSAVYYCARRTVGGYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 9 - DNA, 2026 bp, human
ggatcctctagattgagctttctggggcaggccaggcctgaccttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcctccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccagggaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcctggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgaggaattcagatctggatcc

SEQ ID NO: 10 - protein, 119 aa, murine [sequence omitted]

SEQ ID NO: 11 - protein, 119 aa, artificial sequence
  de-immunized heavy chain variable region
QVQLVQSGAEVKKPGASVKVSCKASGYTATRYTMHWVRQAPGQGLEWIGYINPSRGYTNYAQKFQDRVTITTDKSSSTAYLQMNSLKTEDTAVYYCARYYDDHYCLDYWGQGTTVTVSS

SEQ ID NO: 12 - protein, 119 aa, artificial sequence
  de-immunized heavy chain variable region
QVQLVQSGAEVKKPGASVKVSCKASGYTATRYTMHWVRQAPGQGLEWIGYINPSRGYTNYADSVKGRFTITTDKSSSTAYLQMNSLKTEDTAVYYCARYYDDHYCLDYWGQGTTVTVSS

SEQ ID NO: 13 - protein, 119 aa, artificial sequence
  de-immunized heavy chain variable region
QVQLVQSGAEVKKPGASVKVSCKASGYTATRYTMHWVRQAPGQGLEWIGYINPSRGYTNYNQKFKDRVTITTDKSSSTAYLQMNSLKTEDTAVYYCARYYDDHYCLDYWGQGTTVTVSS

SEQ ID NO: 14 - protein, 119 aa, artificial sequence
  de-immunized heavy chain variable region
QVQLVQSGAEVKKPGASVKVSCKASGYTATRYTMHWVRQAPGQGLEWIGYINPSRGYTNYNQKVKDRFTITTDKSSSTAYLQMNSLKTEDTAVYYCARYYDDHYCLDYWGQGTTVTVSS

SEQ ID NO: 15 - protein, 119 aa, artificial sequence
  de-immunized heavy chain variable region
QVQLVQSGAEVKKPGASVKVSCKASGYTFTRYTMHWVRQAPGQGLEWIGYINPSRGYTNYNQKFKDRVTITTDKSSSTAYLQMNSLKTEDTAVYYCARYYDDHYCLDYWGQGTTVTVSS

SEQ ID NO: 16 - protein, 119 aa, artificial sequence
  de-immunized heavy chain variable region
QVQLVQSGAEVKKPGASVKVSCKASGYTFTRYTMHWVRQAPGQGLEWIGYINPSRGYTNYAQKFQDRVTITTDKSSSTAYLQMNSLKTEDTAVYYCARYYDDHYCLDYWGQGTTVTVSS

SEQ ID NO: 17 - protein, 119 aa, artificial sequence
  de-immunized heavy chain variable region
QVQLVQSGAEVKKPGASVKVSCKASGYTFTRYTMHWVRQAPGQGLEWIGYINPSRGYTNYNQKVKDRFTITTDKSSSTAYLQMNSLKTEDTAVYYCARYYDDHYCLDYWGQGTTVTVSS

SEQ ID NO: 18 - protein, 106 aa, murine [sequence omitted]

SEQ ID NO: 19 - protein, 106 aa, artificial sequence
  de-immunized light chain variable region
QIVLTQSPATLSLSPGERATLTCSASSSASYMNWYQQKPGKAPKRWIYDTSKLASGVPSRFSGSGSGTDYSLTINSLEAEDAATYYCQQWSSNPFTFGQGTKVEIK

SEQ ID NO: 20 - protein, 106 aa, artificial sequence
  de-immunized light chain variable region
QIVLTQSPATLSLSPGERATLTCSASSSVSYMNWYQQKPGKAPKRWIYDTSKLASGVPSRFSGSGSGTDYSLTINSLEAEDAATYYCQQWSSNPFTFGQGTKVEIK

SEQ ID NO: 21 - DNA, 819 bp, artificial sequence
  de-immunized VH expression cassette
aagcttatgaatatgcaaatcctctgaatctacatggtaaatataggtttgtctataccacaaacagaaaaacatgagatcacagttgtctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactcccaggtccagctggtacagtctggggctgaagtcaagaaacctggggcctcagtgaaggtgtcctgcaaggcttctggctacacggctactaggtacacgatgcactgggtaagacaggcgcctggacaaggtttggaatggattggatacattaaccctagccatggatatactaattacgctcagaagttccaggacagggtcacaatcactacagacaaatcttccagcacagcctacttgcaaatgaacagcctgaaaactgaggacaccgcagtctattactgtgcaagatattatgatgatcattactgtctcgactactggggccaaggcaccactgtgacagtctcctcaggtgagtccttacaacctctctcttctattcagcttaaatagattttactgcatttgttgggggggaaatgtgtgtatctgaatttcaggtcatgaaggactagggacaccttgggagtcagaaagggtcattgggagcccgggctgatgcagacagacatcctcagctcccagacttcatggccagagatttataggatcc

SEQ ID NO: 22 - protein, 15 aa, artificial sequence
  signal protein
MGWSCIILFLVATAT

SEQ ID NO: 23 - DNA, 617 bp, artificial sequence
  de-immunized VK expression cassette
aagcttatgaatatgcaaatcctctgaatctacatggtaaatataggtttgtctataccacaaacagaaaaacatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactcccaaattgttctcacccagtctccagcaaccctctctctttctccaggggaacgcgccaccttgacatgcagtgccagctcaagtgcaagttacatgaactggtaccagcagaagcccggcaaagctcccaaaagatggatttatgacacatcaaaactggcttctggagtaccgtctcgcttcagtggcagtgggtctgggaccgattactctctcacaatcaatagtctggaagctgaagatgccgcaacttattactgccagcagtggtcaagtaacccattcacgttcggacaaggtacaaaggtggaaatcaaacgtgagtagaatttaaactttgcttcctcagttggatcc

SEQ ID NO: 24 - protein, 15 aa, artificial sequence
  signal protein
MGWSCIILFLVATAT

SEQ ID NO: 25 - protein, 467 aa, murine
MERHWIFLLLLSVTAGVHSQVQLQQSGAELARPGASVKMSCKASYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - DNA, 1570 bp, murine
gaattcccctctccacagacactgaaaactctgactcaacatggaaaggcctggatctttctactcctgttgtcagtaactgcaggtgtccactcccaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatacttaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctcagcacccacaaaactctcaggtccaaagagacacccacactcatctccatgcttcccttgtataaataaagcacccagcaatgcctgggaccatgtaaaaaaaaaaaaaaaaggaattc

SEQ ID NO: 27 - protein, 235 aa, murine
MDFQVQIFSFLLISASVIISRGQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 28 - DNA, 943 bp, murine
gaattcccaaagacaaaatggattttcaagtgcagattttcagcttcctgctaatcagtgcctcagtcataatatccagaggacaaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaaccgggctgatactgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagctcccagctccatcctatcttcccttctaaggtcttggaggcttccccacaagcgcttaccactgttgcggtgctctaaacctcctcccacctccttctcctcctcctccctttccttggcttttatcatgctaatatttgcagaaaatattcaataaagtgagtctttgccttgaaaaaaaaaaaaaa

SEQ ID NO: 29 - protein, 123 aa, murine
GVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 30 - protein, 110 aa, murine
GVHSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIK

SEQ ID NO: 31 - protein, 12 aa, human
ESKYGPPCPSCP

SEQ ID NO: 32 - protein, 110 aa, human
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 33 - protein, 107 aa, human
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 12 aa, human
ERKCCVECPPCP

SEQ ID NO: 35 - protein, 109 aa, human
APPVAGPSVFLFPPKPKDTLNISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 36 - protein, 107 aa, human; identical to SEQ ID NO: 33

SEQ ID NO: 37 - DNA, 43 bp, artificial sequence
  oligonucleotide
gaagtcaagaaacctggggcctcagtgaaggtgtcctgcaagg

SEQ ID NO: 38 - DNA, 47 bp, artificial sequence
  oligonucleotide
gccccaggtttcttgacttcagccccagactgtaccagctggacctg

SEQ ID NO: 39 - DNA, 31 bp, artificial sequence
  oligonucleotide
tgggtaagacaggcgcctggacaaggtttgg

SEQ ID NO: 40 - DNA, 29 bp, artificial sequence
  oligonucleotide
gtccaggcgcctgtcttacccagtgcatc

SEQ ID NO: 41 - DNA, 48 bp, artificial sequence
  oligonucleotide
aggcgcctgtcttacccagtgcatcgtgtacctagtagccgtgtagcc

SEQ ID NO: 42 - DNA, 43 bp, artificial sequence
  oligonucleotide
caatcagaagttcaaggacagggtcacaatcactacagacaaa

SEQ ID NO: 43 - DNA, 43 bp, artificial sequence
  oligonucleotide
cgctcagaagttccaggacagggtcacaatcactacagacaaa

SEQ ID NO: 44 - DNA, 43 bp, artificial sequence
  oligonucleotide
cgctgacagtgtcaagggcaggttcacaatcactacagacaaa

SEQ ID NO: 45 - DNA, 43 bp, artificial sequence
  oligonucleotide
caatcagaaggtcaaggacaggttcacaatcactacagacaaa

SEQ ID NO: 46 - DNA, 37 bp, artificial sequence
  oligonucleotide
gtccttgaacttctgattgtaattagtatatccacgg

SEQ ID NO: 47 - DNA, 37 bp, artificial sequence
  oligonucleotide
gtcctggaacttctgagcgtaattagtatatccacgg

SEQ ID NO: 48 - DNA, 37 bp, artificial sequence
  oligonucleotide
gcccttgacactgtcagcgtaattagtatatccacgg

SEQ ID NO: 49 - DNA, 37 bp, artificial sequence
  oligonucleotide
gtccttgaccttctgattgtaattagtatatccacgg

SEQ ID NO: 50 - DNA, 35 bp, artificial sequence
  oligonucleotide
agcctgaaaactgaggacaccgcagtctattactg

SEQ ID NO: 51 - DNA, 42 bp, artificial sequence
  oligonucleotide
gtcctcagttttcaggctgttcatttgcaagtaggctgtgct

SEQ ID NO: 52 - DNA, 30 bp, artificial sequence
  oligonucleotide
ccaaggcaccactgtgacagtctcctcagg

SEQ ID NO: 53 - DNA, 30 bp, artificial sequence
  oligonucleotide
cctgaggagactgtcacagtggtgccttgg

SEQ ID NO: 54 - DNA, 24 bp, artificial sequence
  oligonucleotide
ggtgtccactcccaggtccagctg

SEQ ID NO: 55 - DNA, 29 bp, artificial sequence
  oligonucleotide
cagctggacctgggagtggacacctgtgg

SEQ ID NO: 56 - DNA, 37 bp, artificial sequence
  oligonucleotide
gcatgttgaccctgacgcaagcttatgaatatgcaaa

SEQ ID NO: 57 - DNA, 36 bp, artificial sequence
  oligonucleotide
gcgatagctggactgaatggatcctataaatctctg

SEQ ID NO: 58 - DNA, 45 bp, artificial sequence
  oligonucleotide
ccctctctctttctccaggggaacgcgccaccttgacatgcagtg

SEQ ID NO: 59 - DNA, 36 bp, artificial sequence
  oligonucleotide
cctggagaaagagagagggttgctggagactgggtg

SEQ ID NO: 60 - DNA, 48 bp, artificial sequence
  oligonucleotide
catgaactggtaccagcagaagcccggcaaagctcccaaaagatggat

SEQ ID NO: 61 - DNA, 38 bp, artificial sequence
  oligonucleotide
cgggcttctgctggtaccagttcatgtaacttacactt

SEQ ID NO: 62 - DNA, 38 bp, artificial sequence
  oligonucleotide
cttctgctggtaccagttcatgtaacttgcacttgagc

SEQ ID NO: 63 - DNA, 49 bp, artificial sequence
  oligonucleotide
gggtctgggaccgattactctctcacaatcaatagtctggaagctgaag

SEQ ID NO: 64 - DNA, 47 bp, artificial sequence
  oligonucleotide
gtaatcggtcccagacccactgccactgaagcgagacggtactccag

SEQ ID NO: 65 - DNA, 38 bp, artificial sequence
  oligonucleotide
ttcacgttcggacaaggtacaaaggtggaaatcaaacg

SEQ ID NO: 66 - DNA, 38 bp, artificial sequence
  oligonucleotide
ctttgtaccttgtccgaacgtgaatgggttacttgacc

SEQ ID NO: 67 - DNA, 21 bp, artificial sequence
  oligonucleotide
gcggatccagtcgacgaagca

SEQ ID NO: 68 - DNA, 45 bp, artificial sequence
  oligonucleotide
ctgaatggatccaactgaggaagcaaagtttaaattctactcacg

SEQ ID NO: 69 - DNA, 28 bp, artificial sequence
  oligonucleotide
caaattgttctcacccagtctccagcaa

SEQ ID NO: 70 - DNA, 32 bp, artificial sequence
  oligonucleotide
ttgctggagactgggtgagaacaatttgggag

SEQ ID NO: 71 - DNA, 41 bp, artificial sequence
  oligonucleotide
tggagactgggtgagaacaatttgggagtggacacctgtgg

SEQ ID NO: 72 - DNA, 36 bp, artificial sequence
  oligonucleotide
agagagggttgctggagactgggtgagaacaatttg

SEQ ID NO: 73 - DNA, 37 bp, artificial sequence; identical to SEQ ID NO: 56
  oligonucleotide

SEQ ID NO: 74 - DNA, 36 bp, artificial sequence
  oligonucleotide
gcgatagctggactgaatggatccaactgaggaagc

SEQ ID NO: 75 - protein, 122 aa, artificial sequence
  de-immunized OKT3 VH
VSTQVQLVQSGAEVKKPGASVKVSCKASGYTATRYTMHWYRQAPGQGLEWIGYINPSRGYTNYAQKFQQRVTITTDKSSSTAYLQMNSLKTEDTAVYYCARYYDDHYCLDYWGQGTTVTVSG

SEQ ID NO: 76 - protein, 110 aa, artificial sequence
  de-immunized OKT3 VK
GVHSQIVLTQSPATLSLSPGERATLTCSASSSASYMNWYQQKPGKAPKRWIYDTSKLASGVPSRFSGSGSGTDYSLTINSLEAEDAATYYCQQWSSNPFTFGQGTKVEIK

SEQ ID NO: 77 - DNA, 21 bp, artificial sequence
  primer
ttgtgagcggataacaatttc

SEQ ID NO: 78 - DNA, 23 bp, artificial sequence
  primer
gttttcccagtcacgacgttgta

SEQ ID NO: 79 - DNA, 30 bp, artificial sequence
  primer
cttgcagcctccaccaagggcccatccgtc

SEQ ID NO: 80 - DNA, 25 bp, artificial sequence
  primer
cccttggtggaggctgcaagagagg

SEQ ID NO: 81 - DNA, 33 bp, artificial sequence
  primer
gagcctctccctgtctctgggtaaatgagtgcc

SEQ ID NO: 82 - DNA, 35 bp, artificial sequence
  primer
tcatttacccagagacagggagaggctcttctgtg

SEQ ID NO: 83 - DNA, 35 bp, artificial sequence
  primer
tacccggggatccagatctgaattcctcatgtcac